SEQUENCE LISTING (119 sequences)

SEQ ID NO: 1 - protein, 15 aa, Artificial sequence
  Synthetic polypeptide
DYVVGARTFRREKRA

SEQ ID NO: 2 - protein, 15 aa, Artificial sequence
  Synthetic polypeptide
RYWQMRPLFLELLGN

SEQ ID NO: 3 - protein, 15 aa, Artificial sequence
  Synthetic polypeptide
IASIIKPQNTYCVRR

SEQ ID NO: 4 - protein, 15 aa, Artificial sequence
  Synthetic polypeptide
FLELLGNHAQCPYGV

SEQ ID NO: 5 - protein, 15 aa, Artificial sequence
  Synthetic polypeptide
LGNHAQCPYGVLLKT

SEQ ID NO: 6 - protein, 15 aa, Artificial sequence
  Synthetic polypeptide
SPWQVYGFVRACLRR

SEQ ID NO: 7 - protein, 15 aa, Artificial sequence
  Synthetic polypeptide
LWGSRHNERRFLRNT

SEQ ID NO: 8 - protein, 15 aa, Artificial sequence
  Synthetic polypeptide
LQELTWKMSVRDCAW

SEQ ID NO: 9 - protein, 15 aa, Artificial sequence
  Synthetic polypeptide
FYVTETTFQKNRLFF

SEQ ID NO: 10 - protein, 15 aa, Artificial sequence
  Synthetic polypeptide
QSIGIRQHLKRVQLR

SEQ ID NO: 11 - protein, 15 aa, Artificial sequence
  Synthetic polypeptide
IRQHLKRVQLRELSE

SEQ ID NO: 12 - protein, 15 aa, Artificial sequence
  Synthetic polypeptide
LKRVQLRELSEAEVR

SEQ ID NO: 13 - protein, 15 aa, Artificial sequence
  Synthetic polypeptide
RPALLTSRLRFIPKP

SEQ ID NO: 14 - protein, 15 aa, Artificial sequence
  Synthetic polypeptide
PKPDGLRPIVNMDYV

SEQ ID NO: 15 - protein, 15 aa, Artificial sequence
  Synthetic polypeptide
GLRPIVNMDYVVGAR

SEQ ID NO: 16 - protein, 15 aa, Artificial sequence
  Synthetic polypeptide
AWRTFVLRVRAQDPP

SEQ ID NO: 17 - protein, 15 aa, Artificial sequence
  Synthetic polypeptide
LTEVIASIIKPQNTY

SEQ ID NO: 18 - protein, 15 aa, Artificial sequence
  Synthetic polypeptide
IASIIKPQNTYCVRR

SEQ ID NO: 19 - protein, 15 aa, Artificial sequence
  Synthetic polypeptide
RYWQMRPLFLELLGN

SEQ ID NO: 20 - protein, 15 aa, Artificial sequence
  Synthetic polypeptide
FLELLGNHAQCPYGV

SEQ ID NO: 21 - protein, 15 aa, Artificial sequence
  Synthetic polypeptide
AQCPYGVLLKTHCPL

SEQ ID NO: 22 - protein, 15 aa, Artificial sequence
  Synthetic polypeptide
LWGSRHNERRFLRNT

SEQ ID NO: 23 - protein, 15 aa, Artificial sequence
  Synthetic polypeptide
GVGCVPAAEHRLREE

SEQ ID NO: 24 - protein, 15 aa, Artificial sequence
  Synthetic polypeptide
RPALLTSRLRFIPKP

SEQ ID NO: 25 - protein, 15 aa, Artificial sequence
  Synthetic polypeptide
PKPDGLRPIVNMDYV

SEQ ID NO: 26 - protein, 15 aa, Artificial sequence
  Synthetic polypeptide
GLRPIVNMDYVVGAR

SEQ ID NO: 27 - protein, 15 aa, Artificial sequence
  Synthetic polypeptide
IVNMDYVVGARTFRR

SEQ ID NO: 28 - protein, 15 aa, Artificial sequence
  Synthetic polypeptide
DYVVGARTFRREKRA

SEQ ID NO: 29 - protein, 15 aa, Artificial sequence
  Synthetic polypeptide
GASVLGLDDIHRAWR

SEQ ID NO: 30 - protein, 15 aa, Artificial sequence
  Synthetic polypeptide
LGLDDIHRAWRTFVL

SEQ ID NO: 31 - protein, 15 aa, Artificial sequence
  Synthetic polypeptide
LTEVIASIIKPQNTY

SEQ ID NO: 32 - protein, 15 aa, Artificial sequence
  Synthetic polypeptide
IASIIKPQNTYCVRR

SEQ ID NO: 33 - protein, 9 aa, Artificial sequence
  Synthetic polypeptide
RYWQMRPLF

SEQ ID NO: 34 - protein, 9 aa, Artificial sequence
  Synthetic polypeptide
YWQMRPLFL

SEQ ID NO: 35 - protein, 9 aa, Artificial sequence
  Synthetic polypeptide
WQMRPLFLE

SEQ ID NO: 36 - protein, 9 aa, Artificial sequence
  Synthetic polypeptide
QMRPLFLEL

SEQ ID NO: 37 - protein, 9 aa, Artificial sequence
  Synthetic polypeptide
MRPLFLELL

SEQ ID NO: 38 - protein, 9 aa, Artificial sequence
  Synthetic polypeptide
RPLFLELLG

SEQ ID NO: 39 - protein, 9 aa, Artificial sequence
  Synthetic polypeptide
PLFLELLGN

SEQ ID NO: 40 - protein, 10 aa, Artificial sequence
  Synthetic polypeptide
RYWQMRPLFL

SEQ ID NO: 41 - protein, 10 aa, Artificial sequence
  Synthetic polypeptide
YWQMRPLFLE

SEQ ID NO: 42 - protein, 10 aa, Artificial sequence
  Synthetic polypeptide
WQMRPLFLEL

SEQ ID NO: 43 - protein, 10 aa, Artificial sequence
  Synthetic polypeptide
QMRPLFLELL

SEQ ID NO: 44 - protein, 10 aa, Artificial sequence
  Synthetic polypeptide
MRPLFLELLG

SEQ ID NO: 45 - protein, 10 aa, Artificial sequence
  Synthetic polypeptide
RPLFLELLGN

SEQ ID NO: 46 - protein, 9 aa, Artificial sequence
  Synthetic polypeptide
RYWQMRPLF

SEQ ID NO: 47 - protein, 9 aa, Artificial sequence
  Synthetic polypeptide
DYVVGARTF

SEQ ID NO: 48 - protein, 9 aa, Artificial sequence
  Synthetic polypeptide
YVVGARTFR

SEQ ID NO: 49 - protein, 9 aa, Artificial sequence
  Synthetic polypeptide
VVGARTFRR

SEQ ID NO: 50 - protein, 9 aa, Artificial sequence
  Synthetic polypeptide
VGARTFRRE

SEQ ID NO: 51 - protein, 9 aa, Artificial sequence
  Synthetic polypeptide
GARTFRREK

SEQ ID NO: 52 - protein, 9 aa, Artificial sequence
  Synthetic polypeptide
ARTFRREKR

SEQ ID NO: 53 - protein, 9 aa, Artificial sequence
  Synthetic polypeptide
RTFRREKRA

SEQ ID NO: 54 - protein, 10 aa, Artificial sequence
  Synthetic polypeptide
DYVVGARTFR

SEQ ID NO: 55 - protein, 10 aa, Artificial sequence
  Synthetic polypeptide
YVVGARTFRR

SEQ ID NO: 56 - protein, 10 aa, Artificial sequence
  Synthetic polypeptide
VVGARTFRRE

SEQ ID NO: 57 - protein, 10 aa, Artificial sequence
  Synthetic polypeptide
VGARTFRREK

SEQ ID NO: 58 - protein, 10 aa, Artificial sequence
  Synthetic polypeptide
GARTFRREKR

SEQ ID NO: 59 - protein, 10 aa, Artificial sequence
  Synthetic polypeptide
ARTFRREKRA

SEQ ID NO: 60 - protein, 9 aa, Artificial sequence
  Synthetic polypeptide
IASIIKPQN

SEQ ID NO: 61 - protein, 9 aa, Artificial sequence
  Synthetic polypeptide
ASIIKPQNT

SEQ ID NO: 62 - protein, 9 aa, Artificial sequence
  Synthetic polypeptide
SIIKPQNTY

SEQ ID NO: 63 - protein, 9 aa, Artificial sequence
  Synthetic polypeptide
IIKPQNTYC

SEQ ID NO: 64 - protein, 9 aa, Artificial sequence
  Synthetic polypeptide
IKPQNTYCV

SEQ ID NO: 65 - protein, 9 aa, Artificial sequence
  Synthetic polypeptide
KPQNTYCVR

SEQ ID NO: 66 - protein, 9 aa, Artificial sequence
  Synthetic polypeptide
PQNTYCVRR

SEQ ID NO: 67 - protein, 10 aa, Artificial sequence
  Synthetic polypeptide
IASIIKPQNT

SEQ ID NO: 68 - protein, 10 aa, Artificial sequence
  Synthetic polypeptide
ASIIKPQNTY

SEQ ID NO: 69 - protein, 10 aa, Artificial sequence
  Synthetic polypeptide
SIIKPQNTYC

SEQ ID NO: 70 - protein, 10 aa, Artificial sequence
  Synthetic polypeptide
IIKPQNTYCV

SEQ ID NO: 71 - protein, 10 aa, Artificial sequence
  Synthetic polypeptide
IKPQNTYCVR

SEQ ID NO: 72 - protein, 9 aa, Artificial sequence
  Synthetic polypeptide
ILPQNTYCI

SEQ ID NO: 73 - protein, 9 aa, Artificial sequence
  Synthetic polypeptide
ILPQNTYCV

SEQ ID NO: 74 - protein, 9 aa, Artificial sequence
  Synthetic polypeptide
ILPQNTYCL

SEQ ID NO: 75 - protein, 9 aa, Artificial sequence
  Synthetic polypeptide
IMPQNTYCI

SEQ ID NO: 76 - protein, 9 aa, Artificial sequence
  Synthetic polypeptide
IMPQNTYCV

SEQ ID NO: 77 - protein, 9 aa, Artificial sequence
  Synthetic polypeptide
IMPQNTYCL

SEQ ID NO: 78 - protein, 10 aa, Artificial sequence
  Synthetic polypeptide
SLIKPQNTYI

SEQ ID NO: 79 - protein, 10 aa, Artificial sequence
  Synthetic polypeptide
SLIKPQNTYV

SEQ ID NO: 80 - protein, 10 aa, Artificial sequence
  Synthetic polypeptide
SLIKPQNTYL

SEQ ID NO: 81 - protein, 10 aa, Artificial sequence
  Synthetic polypeptide
SMIKPQNTYI

SEQ ID NO: 82 - protein, 10 aa, Artificial sequence
  Synthetic polypeptide
SMIKPQNTYV

SEQ ID NO: 83 - protein, 10 aa, Artificial sequence
  Synthetic polypeptide
SMIKPQNTYL

SEQ ID NO: 84 - protein, 10 aa, Artificial sequence
  Synthetic polypeptide
ILKPQNTYCI

SEQ ID NO: 85 - protein, 10 aa, Artificial sequence
  Synthetic polypeptide
ILKPQNTYCV

SEQ ID NO: 86 - protein, 10 aa, Artificial sequence
  Synthetic polypeptide
ILKPQNTYCL

SEQ ID NO: 87 - protein, 10 aa, Artificial sequence
  Synthetic polypeptide
IMKPQNTYCI

SEQ ID NO: 88 - protein, 10 aa, Artificial sequence
  Synthetic polypeptide
IMKPQNTYCV

SEQ ID NO: 89 - protein, 10 aa, Artificial sequence
  Synthetic polypeptide
IMKPQNTYCL

SEQ ID NO: 90 - protein, 9 aa, Artificial sequence
  Synthetic polypeptide
ILKPQNTYI

SEQ ID NO: 91 - protein, 9 aa, Artificial sequence
  Synthetic polypeptide
ILKPQNTYV

SEQ ID NO: 92 - protein, 9 aa, Artificial sequence
  Synthetic polypeptide
ILKPQNTYL

SEQ ID NO: 93 - protein, 9 aa, Artificial sequence
  Synthetic polypeptide
IMKPQNTYI

SEQ ID NO: 94 - protein, 9 aa, Artificial sequence
  Synthetic polypeptide
IMKPQNTYV

SEQ ID NO: 95 - protein, 9 aa, Artificial sequence
  Synthetic polypeptide
IMKPQNTYL

SEQ ID NO: 96 - protein, 9 aa, Artificial sequence
  Synthetic polypeptide
VLGARTFRK

SEQ ID NO: 97 - protein, 9 aa, Artificial sequence
  Synthetic polypeptide
VLGARTFRR

SEQ ID NO: 98 - protein, 9 aa, Artificial sequence
  Synthetic polypeptide
VIGARTFRK

SEQ ID NO: 99 - protein, 9 aa, Artificial sequence
  Synthetic polypeptide
VIGARTFRR

SEQ ID NO: 100 - protein, 10 aa, Artificial sequence
  Synthetic polypeptide
YLVGARTFRK

SEQ ID NO: 101 - protein, 10 aa, Artificial sequence
  Synthetic polypeptide
YLVGARTFRR

SEQ ID NO: 102 - protein, 10 aa, Artificial sequence
  Synthetic polypeptide
YIVGARTFRK

SEQ ID NO: 103 - protein, 10 aa, Artificial sequence
  Synthetic polypeptide
YIVGARTFRR

SEQ ID NO: 104 - protein, 9 aa, Artificial sequence
  Synthetic polypeptide
RLFRREKRK

SEQ ID NO: 105 - protein, 9 aa, Artificial sequence
  Synthetic polypeptide
RLFRREKRR

SEQ ID NO: 106 - protein, 9 aa, Artificial sequence
  Synthetic polypeptide
RIFRREKRK

SEQ ID NO: 107 - protein, 9 aa, Artificial sequence
  Synthetic polypeptide
RIFRREKRR

SEQ ID NO: 108 - protein, 10 aa, Artificial sequence
  Synthetic polypeptide
VLGARTFRRK

SEQ ID NO: 109 - protein, 10 aa, Artificial sequence
  Synthetic polypeptide
VLGARTFRRR

SEQ ID NO: 110 - protein, 10 aa, Artificial sequence
  Synthetic polypeptide
VIGARTFRRK

SEQ ID NO: 111 - protein, 10 aa, Artificial sequence
  Synthetic polypeptide
VIGARTFRRR

SEQ ID NO: 112 - protein, 10 aa, Artificial sequence
  Synthetic polypeptide
VLARTFRREK

SEQ ID NO: 113 - protein, 10 aa, Artificial sequence
  Synthetic polypeptide
VLARTFRRER

SEQ ID NO: 114 - protein, 10 aa, Artificial sequence
  Synthetic polypeptide
VIARTFRREK

SEQ ID NO: 115 - protein, 10 aa, Artificial sequence
  Synthetic polypeptide
VIARTFRRER

SEQ ID NO: 116 - protein, 9 aa, Artificial sequence
  Synthetic polypeptide
GLRTFRREK

SEQ ID NO: 117 - protein, 9 aa, Artificial sequence
  Synthetic polypeptide
GLRTFRRER

SEQ ID NO: 118 - protein, 9 aa, Artificial sequence
  Synthetic polypeptide
GIRTFRREK

SEQ ID NO: 119 - protein, 9 aa, Artificial sequence
  Synthetic polypeptide
GIRTFRRER

(19) United States
(12) Patent Application Publication
(10) Pub. No.: US 2023/0134801 A1
(43) Pub. Date: May 4, 2023

(54) MHC RESTRICTED IMMUNOGENIC TELOMERASE REVERSE TRANSCRIPTASE -SPECIFIC PEPTIDES, THEIR COMPLEX CONJUGATES, AND METHODS OF USING THE SAME

(71) Applicant: LINEAGE CELL THERAPEUTICS, INC., Carlsbad, CA (US)

(72) Inventors: Debasish SEN, Carlsbad, CA (US); Kevin P. NISHIMOTO, Carlsbad, CA (US); Casey C. CASE, Carlsbad, CA (US); Jane S. LEBKOWSKI, Carlsbad, CA (US)

(21) Appl. No.: 17/995,703

(22) PCT Filed: Apr. 16, 2021
(86) PCT No.: PCT/US21/27751

§ 371 Date: Oct. 6, 2022

Related U.S. Application Data
(60) Provisional application No. 63/012,001, filed on Apr. 17, 2020.

(51) Int. Cl. A61K 39/00

(52) U.S. Cl. CPC A61K 39/001157; A61K 2039/5154; A61K 2039/605

(57) ABSTRACT

The present disclosure provides immunogenic telomerase reverse transcriptase-derived peptide compositions and methods of their use. By administering to a human subject an effective amount of an immunotherapeutic composition that stimulates an immune response to one or more peptides in the composition, a cell-mediated immune response may be elicited in the subject.

### CROSS-REFERENCE TO RELATED APPLICATION

This application claims the benefit of U.S. Provisional Application No. 63/012,001, filed Apr. 17, 2020, which is incorporated herein by reference in its entirety and for all purposes.

### FIELD

The present disclosure relates to the field of cancer immunotherapy and T cell mediated immune response. More specifically, the present disclosure relates to immunogenic telomerase reverse transcriptase-specific peptide compositions and methods of using the same.

### SEQUENCE LISTING

The instant application contains a Sequence Listing, which has been submitted electronically in ASCII format and is hereby incorporated by reference in its entirety. Said ASCII copy, created on Apr. 12, 2021 is named 058125-503001WO_SEQUENCE_LISTING_ST25.txt and is 25,632 bytes in size.

### BACKGROUND

Antigen-presenting cells such as dendritic cells (DCs), process and present protein antigens in the form of peptide-major histocompatibility complex (MHC) molecules on the cell surface (pMHC). T cells recognize the pMHC complex through a cognate T cell receptor. Upon such recognition, T cells may get activated and modulate an immune response against cells expressing the protein antigen. T cell mediated immune response has been utilized in various cancer immunotherapy approaches. These therapies require the activation and expansion of cancer-specific T cells, which then kill cancer cells by recognizing antigen targets expressed on cancer cells.

Telomerase reverse transcriptase (TERT or hTERT in humans) is the catalytic subunit of the enzyme telomerase, a ribonucleoprotein that synthesizes telomeric DNA. hTERT is an attractive tumor-associated antigen for T cell targeting, as it is substantially upregulated in many different kinds of malignant cells, and plays essential roles in extending the lifespan of tumor cells. hTERT is highly upregulated in acute myeloid leukemia (AML), especially the high risk cytogenetics sub-types and is therefore considered an important leukemia-associated antigen candidate. Telomerase-based cancer immunotherapies have previously been successfully generated and safely administered in solid tumor patients.

Mature DCs are potent antigen-presenting cells that modulate T cell mediated immune responses and can be easily generated from peripheral blood mononuclear cells (PBMCs) and loaded with specific antigens. The injection of mature DCs transfected with peptides, proteins, or genes encoding antigens has elicited antigen-specific anti-tumor responses in preclinical experiments, animal models, and clinical studies. Indeed, DCs modified by putative tumor antigens such as lymphoma idiotype protein, prostate alkaline phosphatase, colorectal antigen peptides and tumor lysates have been administered to patients and detectable anti-tumor immune responses were observed. Additionally, DCs can be transfected with translatable mRNA that allows the host's HLA molecules to preferentially select and present epitopes from the antigen's amino acid repertoire.

AST-VAC1 (also referred to as hTERT-DC in the present diclosure) is an immunotherapeutic product that comprises mature DCs transfected with mRNA encoding hTERT and a lysosomal targeting signal, LAMP, that enhances immunostimulatory activity. Previous in vitro studies demonstrated that autologous hTERT-DC stimulate hTERT-specific T cells from the peripheral blood mononuclear cells (PBMCs) of patients with cancer and use of the LAMP sequence enhanced CD8+ and CD4+ T cell responses. More recently, the AST-VAC1 Phase 2 clinical trial using autologous (i.e. patient-derived) hTERT-expressing DCs demonstrated successful anti-hTERT T cell mediated immunity with early signals of improved disease-free survival in patients with high risk AML (Khoury MD et al., Cancer 2017 Aug. 15; 123 (16):3061-3072).

The robust induction of T cell mediated immune responses against hTERT expressing tumors is thought to be mediated by specific hTERT-pMHC complexes that are recognized by responding T cells. While previous studies have attempted to identify hTERT pMHC complexes thought to be important for T cell recognition, the identity and repertoire of potential immunogenic hTERT peptides remains to be elucidated.

### SUMMARY

In one aspect, the present disclosure provides a method for eliciting a cell-mediated immune response in a human subject, comprising administering to the subject an effective amount of an immunotherapeutic composition that stimulates an immune response to one or more peptides selected from the peptides in Tables 1-5 (SEQ ID NOs:4-119).

In some embodiments, the immunotherapeutic composition comprises antigen-presenting cells that present on their surface a peptide selected from the peptides in Tables 1-5. In some embodiments, the immunotherapeutic composition comprises antigen-presenting cells that present on their surface one or more peptides selected from the peptides in Tables 1-5. In some embodiments, the antigen-presenting cells are transfected or pulsed with mRNA encoding one or more peptides selected from the peptides in Tables 1-5.

In some embodiments, the antigen-presenting cells are dendritic cells, macrophages, B cells, or a combination thereof In some embodiments, the antigen-presenting cells are mature dendritic cells.

In some embodiments, the subject is in need of immunotherapy. In some embodiments, the subject has cancer. In some embodiments, the subject has acute myeloid leukemia (AML).

In some embodiments, the mRNA comprises a construct encoding two or more copies of the one or more peptides selected from the peptides in Tables 1-5.

In some embodiments, the antigen-presenting cells are derived from the subject. In some embodiments, the antigen-presenting cells are derived from pluripotent stem cells. In some embodiments, the pluripotent stem cells are human embryonic stem cells.

In another aspect, the present disclosure provides an immunotherapeutic composition that stimulates an immune response to one or more peptides selected from the peptides in Tables 1-5.

In some embodiments, the immunotherapeutic composition comprises antigen-presenting cells that present on their surface a peptide selected from the peptides in Tables 1-5. In some embodiments, the immunotherapeutic composition comprises antigen-presenting cells that present on their surface one or more peptides selected from the peptides in Tables 1-5. In some embodiments, the antigen-presenting cells are transfected or pulsed with mRNA encoding one or more peptides selected from the peptides in Tables 1-5.

In some embodiments, the antigen-presenting cells are dendritic cells, macrophages, B cells, or a combination thereof In some embodiments, the antigen-presenting cells are mature dendritic cells.

In some embodiments, the mRNA comprises a construct encoding two or more copies of any of the one or more peptides selected from the peptides in Tables 1-5.

In yet another aspect, the present disclosure provides an antigen-presenting cell presenting on its surface one or more peptides selected from the peptides in Tables 1-5.

In yet another aspect, the present disclosure provides an antigen-presenting cell transfected or pulsed with mRNA encoding one or more peptides selected from the peptides in Tables 1-5.

In some embodiments, the antigen-presenting cells are dendritic cells, macrophages, B cells, or a combination thereof In some embodiments, the cell is a dendritic cell.

In some embodiments, the mRNA comprises a construct encoding two or more copies of any of the one or more peptides listed in Tables 1-5.

In some embodiments, the transfection is done by electroporation.

In some embodiments, the mRNA construct further comprises a lysosomal targeting sequence.

In yet another aspect, the present disclosure provides an in vitro complex comprising a MEW Class I molecule complexed with a peptide chosen from the peptides listed in Tables 1-5.

In some embodiments, the MEW Class I molecule is encoded by the HLA-A locus. In some embodiments, the in vitro complex further comprises a conjugated biotin molecule.

In various embodiments described herein, the present disclosure provides, inter alia, immunotherapeutic compositions comprising one or more MEW class I restricted immunogenic peptides derived from hTERT. The present disclosure also identifies and provides immunogenic hTERT-derived peptides that are capable of eliciting a positive T cell immune response in a human subject. hTERT-derived immunogenic peptides are identified in Tables 1-5. The present disclosure also provides methods for eliciting a cell mediated immune response in a human subject in need of cellular immunotherapy.

In an embodiment, the present disclosure provides a method for eliciting an immune response in a human subject, comprising administering to the subject an effective amount of an immunotherapeutic composition that stimulates an immune response to one or more peptides selected from the peptides in Tables 1-5. In certain embodiments, the immunotherapeutic composition comprises antigen presenting cells that present on their surface a peptide selected from the peptides list in Tables 1-5. In some embodiments, the antigen presenting cells are transfected or pulsed with mRNA encoding for one or more peptides selected from the peptides in Tables 1-5. In some embodiments, the mRNA comprises a construct encoding for two or more copies of any of the one or more peptides selected from the peptides in Tables 1-5. In some embodiments, the antigen presenting cells are mature dendritic cells. In certain embodiments, the subject is in need of immunotherapy. In some embodiments, the subject has cancer. In one embodiment, the subject has AML. In some embodiments, the antigen presenting cells are derived from the subject. In other embodiments, the antigen presenting cells are derived from pluripotent stem cells. In one embodiment, the pluripotent stem cells are human embryonic stem cells.

### BRIEF DESCRIPTION OF DRAWINGS

For a fuller understanding of the nature and advantages of the present invention, reference should be had to the following detailed description taken in connection with the accompanying drawings, in which:

FIG. 1A depicts enzyme-linked immunospot (ELISPOT) assay design using hTERT peptide pools 1-3 spanning the entire hTERT protein. FIG. 1B depicts hTERT peptide-specific T cell immune responses to peptide pools 1-3 from FIG. 1A. Cryopreserved PBMCs from patients immunized with modified hTERT expressing DCs were thawed and stimulated for 7 days with 1 μg/mL each of 281 15-mer peptides with 11 amino acid overlaps, covering the entire hTERT protein. The cells were subsequently restimulated for 24 hours with peptide pools 1, 2, and 3 at 2.5 μg/mL of each individual peptides within a pool, and IFN-γ ELISPOT counts per 10⁵ cells were recorded. Data was normalized to control wells with cells that were not restimulated with peptides at day 7. Each point represents the mean of triplicate wells. Red lines indicate Mean±SEM. *−pValue<0.01; ns—not significant.

FIG. 2A shows a diagram of modified hTERT protein construct. Peptide pools were algorithmically derived from the highlighted middle region corresponding to amino acids 377-763. FIG. 2B depicts generation of algorithmically deduced peptide pools. Proteasomal analysis using PaProC (Kuttler, C., et al., J Mol Biol, 2000. 298(3): p. 417-29; Nussbaum, A. K., et al., Immunogenetics, 2001. 53(2): p. 87-94) was first utilized to deduce possible peptides cleaved from hTERT377-763. Then, the binding affinity of HLA-A*03:01 or HLA-A*02:01 for these peptides were deduced from using SYFPEITHI (Rammensee, H., et al., Immunogenetics, 1999. 50(3-4): p. 213-9.) A combined binding and cleavage score was assigned using the MAPPP bioinformatics tool (Hakenberg, J., et al., Appl Bioinformatics, 2003. 2(3): p. 155-8). Scored peptides were ranked into top 50% (Hi) and bottom 50% (Low), and then individually mapped to 15-mers covering hTERT377-763. Note that multiple peptides can be mapped to a single 15-mer and vice-versa. 15-mers covered by “Hi” and “Low” comprised “Pool-Hi” and “Pool-Low”, respectively. All non-covered 15-mers collectively comprised “Pool-non”. FIG. 2C depicts ELISPOT assay design using hTERT peptide “hi” “Low” and “Non” pools 1-3 spanning the entire hTERT protein. Cryopreserved PBMCs from patients immunized with modified hTERT expressing DCs were thawed and stimulated for 7 days with 1 μg/mL each of 94 15-mer peptides with 11 amino acid overlaps, covering hTERT amino acids 377-763. The cells were subsequently restimulated for 24 hours with peptide pools “Hi”, “Low”, or “Non” at 2.5 μg/mL of each individual peptides within a pool, and IFN-γ ELISPOT counts per 10⁵ cells were recorded. FIG. 2D depicts peptide-specific immune responses measured as IFN-γ counts per 10⁵ cells for the algorithmically derived pools for HLA-A*03:01 and HLA-A*02:01. Data was normalized to control wells with cells that were not restimulated with peptides at day 7. Data represent Mean±SEM (N=3 wells per condition).

FIGS. 3A, 3B depict individual hTERT peptide-specific T cell immune responses for HLA-A*02:01. Cryopreserved PBMCs from patients immunized with modified hTERT expressing DCs were thawed and stimulated for 7 days with 1 μg/mL each of 94 peptides, length 15 AA each, and encompassing hTERT amino acids 377-763. The cells were subsequently restimulated for 24 h with 2.5 μg/mL of individual peptides and IFN-γ spots per 10⁵ cells were recorded from an ELISPOT assay. FIG. 3A shows a diagram of modified hTERT protein construct. Peptide pools were algorithmically derived from the highlighted middle region corresponding to amino acids 377-763; individual peptides that were used to restimulate cells are shown in location map below. FIG. 3B depicts IFN-γ ELISPOT counts for individual peptides as indicated. Data represent Mean±SEM (N=3 wells per condition). Data was normalized to control wells with cells that were not restimulated with peptides at day 7.

FIGS. 4A, 4B depict individual hTERT peptide-specific T cell immune responses for HLA-A*03:01. Cryopreserved PBMCs from patients immunized with modified hTERT expressing DCs were thawed and stimulated for 7 days with 1 μg/mL each of 94 peptides, length 15 AA each, and encompassing hTERT amino acids 377-763. The cells were subsequently restimulated for 24 h with 2.5 μg/mL of individual peptides and IFN-γ spots per 10⁵ cells were recorded from an ELISPOT assay. FIG. 4A shows a diagram of modified hTERT protein construct. Peptide pools were algorithmically derived from the highlighted middle region corresponding to amino acids 377-763; individual peptides that were used to restimulate cells are shown in location map below. FIG. 4B depicts IFN-γ ELISPOT counts for individual peptides as indicated. Data represent Mean±SEM (N=3 wells per condition). Data was normalized to control wells with cells that were not restimulated with peptides at day 7.

FIG. 5 depicts binding affinity of hTERT peptides specific for HLA-A*03:01 using Prolmmune™ REVEAL™ assay. Individual 9-10 mers derived from hTERT amino acids 385-399, 637-651 and 729-743 respectively (Table 3) were tested using the Prolmmune™ REVEAL™ assay (ProImmune Inc.) to ascertain the binding affinity to HLA-A*03:01. Binding affinity of these peptides was determined as a binding score normalized to the amount of binding observed for a known positive control peptide to HLA*03:01 (control was assigned the value 100).

FIG. 6 depicts binding affinity of hTERT peptides for HLA-A*02:01 by Prolmmune™ REVEAL™ assay. Individual 9-10 mers derived from hTERT729-743 (Table 3) were tested using the Prolmmune™ REVEAL™ assay (ProImmune Inc.) to ascertain the binding affinity to HLA-A*02:01. Binding affinity of these peptides was determined as a binding score normalized to the amount of binding observed for a known positive control peptide to HLA*02:01 (control was assigned the value 100).

FIG. 7 depicts hTERT-specific peptide-major histocompatibility complex (MHC) molecules (pMHC) as monomers or multimers (e.g. tetramers), optionally comprising conjugated biotin-streptavidin.

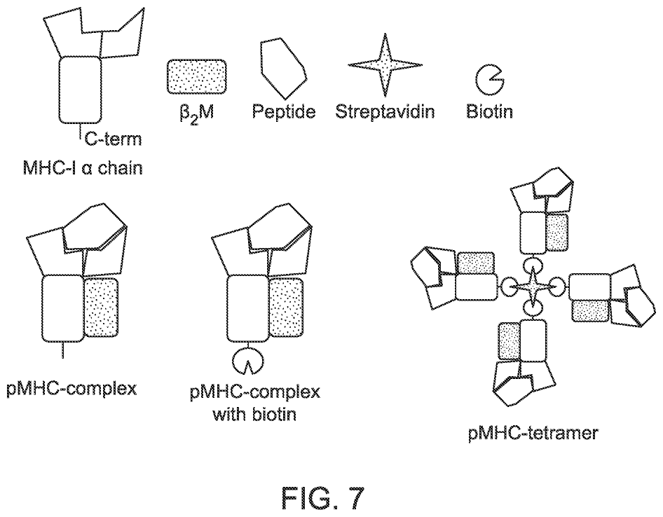

### DETAILED DESCRIPTION

Before the present compositions and methods are described, it is to be understood that the present disclosure is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims For example, features illustrated with respect to one embodiment may be incorporated into other embodiments, and features illustrated with respect to a particular embodiment may be deleted from that embodiment. Thus, the disclosure contemplates that in some embodiments of the disclosure, any feature or combination of features set forth herein can be excluded or omitted. In addition, numerous variations and additions to the various embodiments suggested herein will be apparent to those skilled in the art in light of the instant disclosure, which do not depart from the instant disclosure. In other instances, well-known structures, interfaces, and processes have not been shown in detail in order not to unnecessarily obscure the invention. It is intended that no part of this specification be construed to effect a disavowal of any part of the full scope of the invention. Hence, the following descriptions are intended to illustrate some particular aspects of the disclosure, and not to exhaustively specify all permutations, combinations and variations thereof.

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

All publications, patent applications, patents and other references cited herein are incorporated by reference in their entireties.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

Methods disclosed herein can comprise one or more steps or actions for achieving the described method. The method steps and/or actions may be interchanged with one another without departing from the scope of the present invention. In other words, unless a specific order of steps or actions is required for proper operation of the embodiment, the order and/or use of specific steps and/or actions may be modified without departing from the scope of the present invention.

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a percentages, density, volume and the like, is meant to encompass variations of ±20%, ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

As used herein, the terms “treat,” “treatment,” “therapy,” “therapeutic,” and the like refer to obtaining a desired pharmacologic and/or physiologic effect, including, but not limited to, alleviating, delaying or slowing the progression, reducing the effects or symptoms, preventing onset, inhibiting, ameliorating the onset of a diseases or disorder, obtaining a beneficial or desired result with respect to a disease, disorder, or medical condition, such as a therapeutic benefit and/or a prophylactic benefit. “Treatment,” as used herein, includes any treatment of a disease in a mammal, particularly in a human, and includes: (a) preventing the disease from occurring in a subject, including a subject which is predisposed to the disease or at risk of acquiring the disease but has not yet been diagnosed as having it; (b) inhibiting the disease, i.e., arresting its development; and (c) relieving the disease, i.e., causing regression of the disease. A therapeutic benefit includes eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In some aspects, for prophylactic benefit, treatment or compositions for treatment, including pharmaceutical compositions, are administered to a subject at risk of developing a particular disease, or to a subject reporting one or more of the physiological symptoms of a disease, even though a diagnosis of this disease may not have been made. The methods of the present disclosure may be used with any mammal or other animal. In some aspects, treatment results in a decrease or cessation of symptoms. A prophylactic effect includes delaying or eliminating the appearance of a disease or condition, delaying or eliminating the onset of symptoms of a disease or condition, slowing, halting, or reversing the progression of a disease or condition, or any combination thereof. In some embodiments, the term may refer to both treating and preventing. Treatment includes eliciting a clinically significant response. Treatment also includes prolonging survival as compared to expected survival if not receiving treatment.

The term “subject,” as used herein includes, but is not limited to, humans, non-human primates and non-human vertebrates such as wild, domestic and farm animals including any mammal, such as cats, dogs, cows, sheep, pigs, horses, rabbits, rodents such as mice and rats. In some embodiments, the term “subject,” refers to a male. In some embodiments, the term “subject,” refers to a female.

As used herein, the term “protein” refers to any polymeric chain of amino acids. The terms “peptide” and “polypeptide” can be used interchangeably with the term protein, unless context clearly indicates otherwise, and can also refer to a polymeric chain of amino acids. The term “protein” encompasses native or artificial proteins, protein fragments and polypeptide analogs of a protein sequence. A protein may be monomeric or polymeric. The term “protein” encompasses fragments and variants (including fragments of variants) thereof, unless otherwise contradicted by context.

As used herein, “electroporation” refers to a method for permeabilizing cell membranes by generating membrane pores with electrical stimulation. The applications of electroporation include, but are not limited to, the delivery of DNA, RNA, siRNA, peptides, proteins, antibodies, drugs or other substances to a variety of cells such as mammalian cells, plant cells, yeasts, other eukaryotic cells, bacteria, other microorganisms, and cells from human patients.

The term “in need of treatment” as used herein refers to a judgment made by a caregiver (e.g. physician, nurse, nurse practitioner, or individual in the case of humans; veterinarian in the case of animals, including non-human mammals) that a subj ect requires or will benefit from treatment. This judgment is made based on a variety of factors that are in the realm of a care giver's expertise, but that include the knowledge that the subject is ill, or will be ill, as the result of a condition that is treatable by the disclosed compounds. In specific embodiments described herein, a subject may be in need of immunotherapy.

As used herein, the term “tumor” refers to a mass or lump of tissue that is formed by an accumulation of abnormal cells. A tumor can be benign (i.e., not cancer), malignant (i.e., cancer), or premalignant (i.e., precancerous). The terms “tumor” and “neoplasm” can be used interchangeably. Generally, a cancerous tumor is malignant. As used herein, the term “solid tumor” refers to an abnormal mass of tissue that usually does not contain cysts or liquid areas. Exemplary solid tumors include sarcomas and carcinomas. As used herein, the term “liquid tumors” refers to tumors or cancers present in body fluids such as blood and bone marrow. Exemplary liquid tumors include hematopoietic tumors, such as leukemias and lymphomas, notwithstanding the ability of lymphomas to grow as solid tumors by growing in a lymph node, for example. The term “liquid tumor” can be used interchangeably with the term “blood cancer,” unless context clearly indicates otherwise.

MHC-I and HLA-I are used interchangeably, as are MHC-II and HLA-II.

### Immunotherapeutic Compositions

In some embodiments, the present disclosure provides an immunotherapeutic composition that stimulates an immune response to one or more peptides selected from the peptides in Tables 1-5. Stimulation of an immune response is dependent upon the presence of antigens recognized as foreign by the host immune system. In some embodiments, the immunotherapeutic composition comprises antigen-presenting cells that present on their surface a peptide selected from the peptides in Tables 1-5. In some embodiments, the immunotherapeutic composition comprises antigen-presenting cells that present on their surface a peptide selected from the peptides in Table 1. In some embodiments, the immunotherapeutic composition comprises antigen-presenting cells that present on their surface a peptide selected from the peptides in Table 2. In some embodiments, the immunotherapeutic composition comprises antigen-presenting cells that present on their surface a peptide selected from the peptides in Table 3. In some embodiments, the immunotherapeutic composition comprises antigen-presenting cells that present on their surface a peptide selected from the peptides in Table 4. In some embodiments, the immunotherapeutic composition comprises antigen-presenting cells that present on their surface a peptide selected from the peptides in Table 5. In some embodiments, the immunotherapeutic composition comprises antigen-presenting cells that present on their surface a peptide having the amino acid sequence of any one of SEQ ID NOs:1-119.

Antigen-presenting cells of this invention are often referred to in this disclosure as “dendritic cells”. However, this is not meant to imply any morphological, phenotypic, or functional feature beyond what is explicitly required. The term is used to refer to cells that are phagocytic or can present antigen to T lymphocytes, falling within the general class of monocytes, macrophages, dendritic cells and the like, such as may be found circulating in the blood or lymph, or fixed in tissue sites. Phagocytic properties of a cell can be determined according to their ability to take up labeled antigen or small particulates. The ability of a cell to present antigen can be determined in a mixed lymphocyte reaction.

Dendritic cells are derived from hematopoietic bone marrow progenitor cells. These progenitor cells initially transform into immature dendritic cells. These cells are characterized by high endocytic activity and low T-cell activation potential. Immature dendritic cells constantly sample the surrounding environment for pathogens such as viruses and bacteria. This is done through pattern recognition receptors such as the toll-like receptors. Toll-like receptors recognize specific chemical signatures found on subsets of pathogens. Once immature dendritic cells have come into contact with a presentable antigen, they become activated into mature dendritic cells and begin to migrate to a lymph node. Immature dendritic cells phagocytose pathogens and degrade their proteins into small pieces and upon maturation present those fragments at their cell surface using MHC molecules. Simultaneously, they upregulate cell-surface receptors that act as co-receptors in T-cell activation such as CD80 (B7.1), CD86 (B7.2), and CD40 greatly enhancing their ability to activate T-cells. They also upregulate CCR7, a chemotactic receptor that induces the dendritic cell to travel through the blood stream to the spleen or through the lymphatic system to a lymph node. Here they act as antigen-presenting cells: they activate helper T-cells and killer T-cells as well as B-cells by presenting them with antigens derived from the pathogen, alongside non-antigen specific costimulatory signals. Dendritic cells can also induce T-cell tolerance (unresponsiveness). Certain C-type lectin receptors on the surface of dendritic cells, some functioning as pattern recognition receptors, help instruct dendritic cells as to when it is appropriate to induce immune tolerance rather than lymphocyte activation.

Every helper T-cell is specific to one particular antigen. Only professional antigen-presenting cells (macrophages, B lymphocytes, and dendritic cells) are able to activate a resting helper T-cell when the matching antigen is presented. However, in non-lymphoid organs, macrophages and B cells can only activate memory T cells whereas dendritic cells can activate both memory and naive T cells, and are the most potent of all the antigen-presenting cells. In the lymph node and secondary lymphoid organs, all three cell types can activate naive T cells. Whereas mature dendritic cells are able to activate antigen-specific naive CD8+ T cells, the formation of CD8+ memory T cells requires the interaction of dendritic cells with CD4+ helper T cells. This help from CD4+ T cells additionally activates the matured dendritic cells and licenses them to efficiently induce CD8+ memory T cells, which are also able to be expanded a second time. For this activation of dendritic cells, concurrent interaction of all three cell types, namely CD4+ T helper cells, CD8+ T cells and dendritic cells, seems to be required.

Mature dendritic cells (mDCs) probably arise from monocytes, white blood cells which circulate in the body and, depending on the right signal, can turn into either dendritic cells or macrophages. The monocytes in turn are formed from stem cells in the bone marrow. Monocyte-derived dendritic cells can be generated in vitro from peripheral blood mononuclear cell (PBMCs). Plating of PBMCs in a tissue culture flask permits adherence of monocytes. Treatment of these monocytes with interleukin 4 (IL-4) and granulocyte-macrophage colony stimulating factor (GM-CSF) leads to differentiation to immature dendritic cells (iDCs) in about a week. Subsequent treatment with tumor necrosis factor (TNF) further differentiates the immature dendritic cells (imDCs) into mature dendritic cells. Monocytes can be induced to differentiate into dendritic cells by a self-peptide Ep1.B derived from apolipoprotein E. These are primarily tolerogenic plasmacytoid dendritic cells.

Immature dendritic cells may be matured to mDCs by contacting the imDC with a suitable maturation cocktail comprising a plurality of exogenous cytokines. The maturation cocktail may comprise GM-CSF. In an embodiment, the maturation cocktail includes GM-CSF, TNFα, IL-1β, IFNγ, and PGE2. In an embodiment, the maturation cocktail includes GM-CSF, TNFα, IL-1β, IFNγ, PGE2 and CD40L. In an embodiment, the maturation cocktail includes GM-CSF, TNFα, IL-I β, IFNγ, PGE2, POLY LC, and IFNα. In an embodiment, the maturation cocktail includes GM-CSF, TNFα, IL-I β, IFNγ, POLY LC, and IFNα. In an embodiment, the maturation cocktail includes GM-CSF, TNFα, IL-1β, IFNγ, POLY LC, IFNα, and CD40L. In an embodiment, the maturation cocktail includes TNFα, IL-I β, PGE2 and IL-6. In an embodiment, the maturation cocktail includes GM-CSF, IL-1β, PGE2, and, IFNγ. In an embodiment, the maturation cocktail includes GM-CSF, TNFα, PGE2, and, IFNγ. In an embodiment, the maturation cocktail includes GM-CSF, IL-1β, IFNγ and CD40L. In some embodiments, ligands to one or more cytokine receptors may be used in place of, and/or in addition to the cytokine. Other methods, known in the art, may be used to mature imDC to mDC. Non-limiting examples include contacting imDC with lipopolysaccharide (LPS), contacting the imDC with CpG containing oligonucleotides, or injecting the imDC into an area of inflammation within a subject.

In some embodiments, antigen-presenting cells may include, but are not necessarily limited to, dendritic cells, macrophages, and B cells. In some embodiments, the antigen-presenting cells are dendritic cells. In embodiments, the antigen-presenting cells are mature dendritic cells. In embodiments, the antigen-presenting cells are macrophages. In embodiments, the antigen-presenting cells are B cells.

In some embodiments, the antigen, when administered to a mammalian subject, raises an immune response to a pathogen. In embodiments, the pathogen is bacterial. In embodiments, the pathogen is viral. In embodiments, the pathogen is fungal. In embodiments, the pathogen is protozoan. In embodiments, the pathogen is cancerous.

In some embodiments, the antigenic protein is expressed on the outer surface of the pathogen; while in other embodiments, the antigen may be a non-surface antigen, e.g., useful as a T-cell epitope. The immunogen may elicit an immune response against a pathogen (e.g. a bacterium, a virus, a fungus or a parasite) but, in some other embodiments, it elicits an immune response against an allergen or a tumor antigen. The immune response may comprise an antibody response (usually including IgG) and/or a cell-mediated immune response. A polypeptide immunogen will typically elicit an immune response that recognizes the corresponding pathogen (or allergen or tumor) polypeptide, but in some embodiments, the polypeptide may act as a mimotope to elicit an immune response that recognizes a saccharide. The immunogen will typically be a surface polypeptide e.g. an adhesin, a hemagglutinin, an envelope glycoprotein, a spike glycoprotein, etc.

In some embodiments, the immunotherapeutic composition comprises antigen-presenting cells that present on their surface a peptide selected from the peptides in Tables 1-5.

In some embodiments, the antigen-presenting cells are derived from the subject to be treated. In some embodiments, the antigen-presenting cells are allogeneic. In some embodiments, the antigen-presenting cells are derived from stem cells. In some embodiments, the antigen-presenting cells are derived from pluripotent stem cells.

As used herein, “pluripotent stem cells” refers to cells that may be derived from any source and that are capable, under appropriate conditions, of producing primate progeny of different cell types that are derivatives of all of the 3 germinal layers (endoderm, mesoderm, and ectoderm). Primate pluripotent stem cells (pPS) may have the ability to form a teratoma in 8-12 week old SCID mice and/or the ability to form identifiable cells of all three germ layers in tissue culture. Included in the definition of primate pluripotent stem cells are embryonic cells of various types including human embryonic stem (hES) cells, (see, e.g., Thomson et al. (1998) Science 282: 1145) and human embryonic germ (hEG) cells (see, e.g., Shamblott et al., (1998) Proc. Natl. Acad. Sci. USA 95: 13726,); embryonic stem cells from other primates, such as Rhesus stem cells (see, e.g., Thomson et al., (1995) Proc. Natl. Acad. Sci. USA 92:7844), marmoset stem cells (see, e.g., (1996) Thomson et al., Biol. Reprod. 55:254,), stem cells created by nuclear transfer technology (U.S. Patent Application Publication No. 2002/0046410), as well as induced pluripotent stem cells (see, e.g. Yu et al., (2007) Science 318:5858); Takahashi et al., (2007) Cell 131(5):861). As used herein, “undifferentiated primate pluripotent stem cells” refers to a cell culture where a substantial proportion of primate pluripotent stem cells and their derivatives in the population display morphological characteristics of undifferentiated cells. It is understood that colonies of undifferentiated cells within the population may be surrounded by neighboring cells that are partly differentiated.

As used herein, “genetically altered”, “genetically modified”, “transfected”, or “genetically transformed” refer to a process where a polynucleotide has been transferred into a cell by any suitable means of artificial manipulation, or where the cell is a progeny of the originally altered cell and has inherited the polynucleotide. The polynucleotide will often comprise a transcribable sequence encoding a protein of interest, which enables the cell to express the protein at an elevated level or may comprise a sequence encoding a molecule such as siRNA or antisense RNA that affects the expression of a protein (either expressed by the unmodified cell or as the result of the introduction of another polynucleotide sequence) without itself encoding a protein. The genetic alteration is said to be “inheritable” if progeny of the altered cell have the same alteration.

For details about methods of differentiating primate pluripotent stem cells, reference is made to PCT Application No. PCT/US2009/038442, the contents of which are incorporated herein by reference in their entirety.

In specific embodiments, the pluripotent stem cells are human embryonic stem cells.

In certain embodiments the present disclosure provides compositions for stimulating an immune response to an antigen comprising contacting a cell according to the invention with an antigen. The antigen may be comprised of a protein or peptide or alternatively it may be comprised of a nucleic acid e.g. DNA, RNA. Where the antigen is a protein or peptide the dendritic cell or other antigen-presenting cell will take up the protein or peptide and process it for presentation in the context of the MHC. Typically processing includes proteolysis so that the antigen will fit in the MHC groove. Where the antigen is a protein the dendritic cell may be an immature dendritic cell. Where the antigen is a peptide fragment of a full length protein the dendritic cell may be a mature dendritic cell. Where the antigen is a nucleic acid the invention contemplates using any means known in the art for transporting the nucleic acid across the cell membrane for delivery into the cytoplasm.

In embodiments the cells may be electroporated or “pulsed” to allow the nucleic acid to cross the cell membrane. In some embodiments where electroporation is used to contact the cell with an antigen a suitable cell may be an immature dendritic cell. In other embodiments where electroporation is used to contact the cell with an antigen a suitable cell may be a mature dendritic cell. The cells may be electroporated using Gene Pulse Xcell (Bio-Rad Laboratories, Hercules, Calif.) with the following parameters: 300V, 15 OuF, and 100 Ohms. Protein expression levels may be determined by flow cytometry or western blot methods. Where the electroporated cell is an immature dendritic cell, the cell may be contacted with a maturation cocktail such that the immature dendritic cells mature into mature dendritic cells. In another embodiment a viral vector may be used to transport the nucleic acid encoding the antigen into the cell, e.g., a mature dendritic cell, an immature dendritic cell. Where a viral vector is used to contact the cell with an antigen, a suitable cell may be an immature dendritic cell. Examples of suitable viral vectors include adenoviral vectors and pox viral vectors. In other embodiments commercially available transfection reagents may be used to transport the nucleic acid encoding the antigen into the cell. Suitable examples include cationic lipid formulations such as LIPOFECTAMINE®.

In some embodiments, the antigen-presenting cells are transfected or pulsed with mRNA encoding one or more peptides selected from the peptides in Tables 1-5. In some embodiments, the antigen-presenting cells are transfected or pulsed with DNA encoding one or more peptides selected from the peptides in Tables 1-5. In some embodiments, the antigen-presenting cells can be genetically modified to express the peptide.

In some embodiments, the mRNA comprises a construct encoding two or more copies of any of the one or more peptides selected from the peptides in Tables 1-5.

In some embodiments, the subject may be in need of immunotherapy. In specific embodiments, the subject has cancer. In some embodiments, the cancer may be kidney cancer, renal cancer, urinary bladder cancer, prostate cancer, uterine cancer, breast cancer, cervical cancer, ovarian cancer, lung cancer, liver cancer, stomach cancer, colon cancer, rectal cancer, oral cavity cancer, pharynx cancer, pancreatic cancer, thyroid cancer, melanoma, skin cancer, head and neck cancer, brain cancer, hematopoietic cancer, leukemia, lymphoma, bone cancer, or sarcoma.

Exemplary tumor proteins or tumor antigens include products of mutated oncogenes, products of mutated tumor suppressor genes, products of mutated genes other than oncogenes or tumor suppressors, tumor antigens produced by oncogenic viruses, altered cell surface glycoproteins, oncofetal antigens, and others. Tumor proteins or tumor antigens also include immune regulatory molecules, such as immune checkpoint inhibitors and immune stimulatory molecules. Tumor antigens further include altered cell surface glycolipids. In some aspects, the tumor protein or tumor antigen encoded by transgenes included in second polynucleotides of nucleic acid molecules provided herein is a kidney cancer, renal cancer, urinary bladder cancer, prostate cancer, uterine cancer, breast cancer, cervical cancer, ovarian cancer, lung cancer, liver cancer, stomach cancer, colon cancer, rectal cancer, oral cavity cancer, pharynx cancer, pancreatic cancer, thyroid cancer, melanoma, skin cancer, head and neck cancer, brain cancer, hematopoietic cancer, leukemia, lymphoma, bone cancer, or sarcoma protein. Exemplary tumor proteins or tumor antigens include KRAS, NRAS, HRAS, HER2, BRCA1, BRCA2, carcinoembryonic antigen (CEA), MUC1, guanylyl-cyclase C, NY-ESO-1, melanoma-associated antigen (e.g., MAGE-1, MAGE-3), p53, survivin, alphafetoprotein (AFP), CA-125, epithelial tumor antigen (ETA), tyrosinase, prostate-specific antigen (PSA), prostate-specific membrane antigen (PSMA), prostate stem cell antigen (PSCA), human aspartyl (asparaginyl) β-hydroxylase (HAAH), EphA2, and others.

In some embodiments, the cancer is leukemia. In embodiments, the cancer is acute myeloid leukemia (AML).

### Antigen-Presenting Cells and Complexes

In some embodiments, the present disclosure provides an antigen-presenting cell presenting on its surface a peptide selected from the peptides in Tables 1-5. In some embodiments, the antigen-presenting cell is transfected or pulsed with mRNA encoding one or more peptides selected from the peptides in Tables 1-5. In some embodiments, the mRNA comprises a construct encoding two or more copies of any of the one or more peptides listed in Tables 1-5. As described herein, antigen-presenting cells may be dendritic cells, macrophages, B cells, or a combination thereof In embodiments, the antigen-presenting cell is a dendritic cell.

In some embodiments, the antigen-presenting cell is transfected by electroporation, as described in detail elsewhere herein.

In some embodiments, the mRNA construct further comprises a lysosomal targeting sequence.

Mammalian lysosomal enzymes are synthesized in the cytosol and traverse the ER where they are glycosylated with N-linked, high mannose type carbohydrate. In the Golgi, the high mannose carbohydrate is modified on lysosomal proteins by the addition of mannose-6-phosphate (M6P) which targets these proteins to the lysosome. The M6P-modified proteins are delivered to the lysosome via interaction with either of two M6P receptors. The most favorable form of modification is when two M6Ps are added to a high mannose carbohydrate.

The majority of soluble acid hydrolases are modified with mannose 6-phosphate (M6P) residues, allowing their recognition by M6P receptors in the Golgi complex and ensuing transport to the endosomal/lysosomal system. Other soluble enzymes and non-enzymatic proteins are transported to lysosomes in an M6P-independent manner mediated by alternative receptors such as the lysosomal integral membrane protein LIMP-2 or sortilin. Sorting of cargo receptors and lysosomal transmembrane proteins requires sorting signals present in their cytosolic domains. These signals include dileucine-based motifs, DXXLL or [DE]XXXL[LI], and tyrosine-based motifs, YXXO, which interact with components of clathrin coats such as GGAs or adaptor protein complexes. In addition, phosphorylation and lipid modifications regulate signal recognition and trafficking of lysosomal membrane proteins. The complex interaction of both luminal and cytosolic signals with recognition proteins guarantees the specific and directed transport of proteins to lysosomes.

The available data suggest the following sequence of events in the intracellular transport of MHC class II molecules: MHC class II molecules with the invariant chain are assembled in the endoplasmic reticulum and transported through the Golgi in common with other membrane proteins including MHC class I. The molecules are then targeted to specific endosomal/lysosomal organelles by an unknown mechanism, segregating from the MHC class I molecules which follow a constitutive route to the cell surface. In the endocytotic/lysosomal route, the invariant chain is removed from MHC class II by proteases acting in an acidic environment. At the same time, antigenic fragments of proteins that have entered the endocytic/lysosomal pathway are generated by these proteases and the resulting peptides bind to the class II molecules and are carried to the cell surface.

Antigen-presenting cells and dendritic cells, in particular, present antigen to the lymphocytes in the context of the appropriate major histocompatibility complex (MHC) and thus provide the initial stimulus for mounting the adaptive immune response. In some embodiments, the present disclosure provides an in vitro complex comprising a MHC Class I molecule complexed with a peptide chosen from the peptides listed in Tables 1-5.

In vitro complexes can be selectively isolated and can be delivered into cells using a variety of methods, as described in more detail elsewhere herein. For in vivo delivery, a transfection complex (transfection reagent in association with the nucleic acid to be delivered) should be small, less than 100 nm in diameter, such less than 50 nm. Even smaller complexes, less than 20 nm or less than 10 nm may also be used. Transfection complexes larger than 100 nm have very little access to cells other than blood vessel cells in vivo. In vitro complexes are also positively charged. This positive charge is necessary for attachment of the complex to the cell and for membrane fusion, destabilization or disruption. Cationic charge on in vivo transfection complexes leads to adverse serum interactions and therefore poor bioavailability. Near neutral or negatively charged complexes would have better in vivo distribution and targeting capabilities. However, in vitro transfection complexes associate with nucleic acid via charge-charge (electrostatic) interactions. Negatively charged polymers and lipids do not interact with negatively charged nucleic acids. Further, these electrostatic complexes tend to aggregate or fall apart when exposed to physiological salt concentrations or serum components. Finally, transfection complexes that are effective in vitro are often toxic in vivo. Polymers and lipids used for transfection disrupt or destabilize cell membranes. Balancing this activity with nucleic acid delivery is more easily attained in vitro than in vivo.

In some embodiments, the MHC Class I molecule of the in vitro complex is encoded by the HLA-A locus.

For targeting complexes to specific target cells, tissues, or specific cell types, targeting groups, or ligands, may be used. Targeting groups enhance the association of molecules with a target cell. Thus, targeting groups can enhance the pharmacokinetic or biodistribution properties of a conjugate to which they are attached to improve cellular distribution and cellular uptake of the conjugate. One or more targeting groups can be linked to the membrane active polymer either directly or via a linkage with a spacer. Binding of a targeting group, such as a ligand, to a cell or cell receptor may initiate endocytosis. Targeting groups may be monovalent, divalent, trivalent, tetravalent, or have higher valency. Targeting groups may be selected from the group comprising: compounds with affinity to cell surface molecule, cell receptor ligands, and antibodies, antibody fragments, and antibody mimics with affinity to cell surface molecules. A preferred targeting group comprises a cell receptor ligand. A variety of ligands have been used to target drugs and genes to cells and to specific cellular receptors. Cell receptor ligands may be selected from the group comprising: carbohydrates, glycans, saccharides (including, but not limited to: galactose, galactose derivatives, mannose, and mannose derivatives), vitamins, folate, biotin, aptamers, and peptides (including, but not limited to: RGD-containing peptides, insulin, EGF, and transferrin). Examples of targeting groups include those that target the asialoglycoprotein receptor by using asialoglycoproteins or galactose residues. For example, liver hepatocytes contain ASGP Receptors. Therefore, galactose-containing targeting groups may be used to target hepatocytes. Galactose containing targeting groups include, but are not limited to: galactose, N-acetylgalactosamine, oligosaccharides, and saccharide clusters (such as: Tyr-Glu-Glu-(aminohexyl GalNAc)3, lysine-based galactose clusters, and cholane-based galactose clusters). Further suitable conjugates can include oligosaccharides that can bind to carbohydrate recognition domains (CRD) found on the asialoglycoprotein-receptor (ASGP-R). Example conjugate moieties containing oligosaccharides and/or carbohydrate complexes are provided in U.S. Pat. No. 6,525,031.

In some embodiments, the MHC:peptide complexes can be coupled to the surface of polystyrene particles (microbeads) by biotin:streptavidin biochemistry, as described in the examples. This system permits the exact control of the MHC density on antigen-presenting cells, which allows for selectively eliciting high- or low-avidity antigen-specific T cell responses with high efficiency from blood samples.

In some embodiments, the in vitro complex may further comprise a conjugated biotin molecule.

### Methods for Eliciting Cell-Mediated Immune Responses

In other embodiments, the present disclosure also provides methods for eliciting a cell-mediated immune response in a human subject, comprising administering to the subject an effective amount of an immunotherapeutic composition according to the present disclosure that stimulates an immune response to one or more peptides selected from the peptides in Tables 1-5.

Any route of administration can be included in methods provided herein. In some aspects, nucleic acid molecules, compositions, and pharmaceutical compositions provided herein are administered intramuscularly, subcutaneously, intradermally, transdermally, intranasally, orally, sublingually, intravenously, intraperitoneally, topically, by aerosol, or by a pulmonary route, such as by inhalation or by nebulization, for example. In some embodiments, the pharmaceutical compositions described are administered systemically. Suitable routes of administration include, for example, rectal, vaginal, transmucosal, or intestinal administration; parenteral delivery, including intramedullary injections, as well as intrathecal, direct intraventricular, intravenous, intraperitoneal, or intranasal. In particular embodiments, the intramuscular administration is to a muscle selected from the group consisting of skeletal muscle, smooth muscle and cardiac muscle. In some embodiments, the pharmaceutical composition is administered intravenously.

As used herein, the term “effective amount” or “therapeutically effective amount” refers to that amount of a nucleic acid molecule, composition, or pharmaceutical composition described herein that is sufficient to effect the intended application, including but not limited to inducing an immune response and/or disease treatment, as defined herein. The therapeutically effective amount may vary depending upon the intended application (e.g., inducing an immune response, treatment, application in vivo), or the subject or patient and disease condition being treated, e.g., the weight and age of the subject, the species, the severity of the disease condition, the manner of administration and the like, which can readily be determined by one of ordinary skill in the art. The term also applies to a dose that will induce a particular response in a target cell. The specific dose will vary depending on the particular nucleic acid molecule, composition, or pharmaceutical composition chosen, the dosing regimen to be followed, whether it is administered in combination with other compounds, timing of administration, the tissue to which it is administered, and the physical delivery system in which it is carried.

Any type of immune response can be induced using the methods provided herein, including adaptive and innate immune responses. In one aspect, immune responses induced using the methods provided herein include an antibody response, a cellular immune response, or both an antibody response and a cellular immune response.

In some embodiments, the immunotherapeutic composition comprises antigen-presenting cells that present on their surface a peptide selected from the peptides in Tables 1-5. In some embodiments, the antigen-presenting cells are derived from the subject. In some embodiments, the antigen-presenting cells are derived from pluripotent stem cells. In some embodiments, the pluripotent stem cells are human embryonic stem cells, as described elsewhere herein.

In some embodiments, the antigen-presenting cells are transfected or pulsed with mRNA encoding one or more peptides selected from the peptides in Tables 1-5, as described elsewhere herein. In some embodiments, the mRNA comprises a construct encoding two or more copies of any of the one or more peptides selected from the peptides in Tables 1-5.

In some embodiments, the antigen-presenting cells may be dendritic cells, macrophages, B cells, or a combination thereof, as described elsewhere herein. In specific embodiments, the antigen-presenting cells are mature dendritic cells.

In some embodiments, the subject is in need of immunotherapy. In some embodiments, the subject has cancer. In some embodiments, the cancer may be kidney cancer, renal cancer, urinary bladder cancer, prostate cancer, uterine cancer, breast cancer, cervical cancer, ovarian cancer, lung cancer, liver cancer, stomach cancer, colon cancer, rectal cancer, oral cavity cancer, pharynx cancer, pancreatic cancer, thyroid cancer, melanoma, skin cancer, head and neck cancer, brain cancer, hematopoietic cancer, leukemia, lymphoma, bone cancer, or sarcoma. In specific embodiments, the cancer is leukemia, such as has acute myeloid leukemia (AML).

In certain embodiments the invention provides a method of stimulating an immune response to an antigen comprising contacting a cell according to the invention, e.g., a DC differentiated from pPS cells, with an antigen. The antigen may be comprised of a protein or peptide or alternatively it may be comprised of a nucleic acid e.g. DNA, RNA. Where the antigen is a protein or peptide the dendritic cell will take up the protein or peptide and process it for presentation in the context of the MEW. Typically processing includes proteolysis so that the antigen will fit in the MHC groove. Where the antigen is a protein the DC cell may be an imDC. Where the antigen is a peptide fragment of a full length protein the DC may be a mDC. Where the antigen is a nucleic acid the invention contemplates using any means known in the art for transporting the nucleic acid across the cell membrane for delivery into the cytoplasm. In one embodiment the cells may be electroporated to allow the nucleic acid to cross the cell membrane. In some embodiments where electroporation is used to contact the cell with an antigen a suitable cell may be an imDC. In other embodiments where electroporation is used to contact the cell with an antigen a suitable cell may be an mDC. The cells may be electroporated using Gene Pulse Xcell (Bio-Rad Laboratories, Hercules, Calif.) with the following parameters: 300V, 15 OuF, and 100 Ohms. Protein expression levels may be determined by flow cytometry or western blot methods. Where the electroporated cell is an imDC the cells may be contacted with a maturation cocktail as described herein such that the imDC mature into mDC.

In another embodiment a viral vector may be used to transport the nucleic acid encoding the antigen into the cell, e.g., a mDC, an imDC. Where a viral vector is used to contact the cell with an antigen a suitable cell may be imDC. Examples of suitable viral vectors include adenoviral vectors and pox viral vectors. In other embodiments commercially available transfection reagents may be used to transport the nucleic acid encoding the antigen into the cell. Suitable examples include cationic lipid formulations such as Lipofectamine®.

The invention contemplates using antigens from any source. Thus the antigen may be a tumor antigen such as human telomerase reverse transcriptase (hTERT) as described in the examples, or an antigen expressed by infectious agent such as a virus, a bacterium, or a parasite. The mDC may then be contacted, either in vivo or in vitro, with an immunologically competent cell such as a lymphocyte. The immune response of the lymphocyte may be monitored by measuring cell proliferation of the immunologically competent cell (e.g., by 3H thymidine incorporation) and/or cytokine production (e.g. IL-2, IFN, IL-6, IL-12) by either the mDC or the immunologically competent cell. These studies may be useful in tailoring the type and extent of the immune response to the antigen. These studies may also be useful in selecting the best epitope of the antigen for eliciting the most appropriate immune response. The immune response may be stimulated in vitro or in vivo using an appropriate animal model.

To determine the suitability of cell compositions for therapeutic administration, the cells can first be tested in a suitable animal model. Suitable animal models may include a mouse with a humanized immune system. See, e.g., Goldstein (2008) AIDS Res Ther 5(1):3. mDC primed with a specific antigen may be administered to an animal to determine whether or not the animal is able to mount a specific immune response to the antigen. The animal and the DC may be matched or partially matched at the MHC I locus. Dosing, administration and formulation of the antigen and of the cells may be studied to tailor the immune response to the antigen and migration of the administered cells within the lymphatic system may be monitored. The extent of the immune response may be characterized in terms of cytokine production as well as lymphocyte proliferation in response to the antigen. The animal may be monitored for an antibody response against the antigen as well as for any atypical immune reaction, e.g. hypersensitivity, autoimmune reaction. The antibody generated may be isolated for use as a research reagent or therapeutic agent.

Immature dendritic cells are known to induce antigen specific tolerance, see, e.g., Cools et al., (2007) J Leukoc Biol 82(6): 1365. Thus imDC, as described herein, may be used to induce tolerance within a subject. The imDC cells may be contacted with antigen, e.g., a protein or peptide antigen or a nucleic acid encoding an antigen as described above. The cells may then be administered to a subject to induce tolerance in the subject. Alternatively, the imDC may be matured into mDC and used to stimulate an immune response.

In embodiments described herein, the peptide (e.g., presented at the surface of the cell, complexed with MHC, etc.) is a peptide having the amino acid sequence of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, or SEQ ID NO:119. Similarly, in embodiments described herein, a nucleic acid may encode a peptide having the amino acid sequence of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, or SEQ ID NO:119. In embodiments described herein, an mRNA (e.g., an mRNA introduced into a cell) may encode a peptide having the amino acid sequence of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, or SEQ ID NO:119.

In some embodiments, the peptide has the amino acid sequence of SEQ ID NO:1. In some embodiments, the peptide has the amino acid sequence of SEQ ID NO:2. In some embodiments, the peptide has the amino acid sequence of SEQ ID NO:3. In some embodiments, the peptide has the amino acid sequence of SEQ ID NO:4. In some embodiments, the peptide has the amino acid sequence of SEQ ID NO:5. In some embodiments, the peptide has the amino acid sequence of SEQ ID NO:6. In some embodiments, the peptide has the amino acid sequence of SEQ ID NO:7. In some embodiments, the peptide has the amino acid sequence of SEQ ID NO:8. In some embodiments, the peptide has the amino acid sequence of SEQ ID NO:9. In some embodiments, the peptide has the amino acid sequence of SEQ ID NO:10. In some embodiments, the peptide has the amino acid sequence of SEQ ID NO:11. In some embodiments, the peptide has the amino acid sequence of SEQ ID NO:12. In some embodiments, the peptide has the amino acid sequence of SEQ ID NO:13. In some embodiments, the peptide has the amino acid sequence of SEQ ID NO:14. In some embodiments, the peptide has the amino acid sequence of SEQ ID NO:15. In some embodiments, the peptide has the amino acid sequence of SEQ ID NO:16. In some embodiments, the peptide has the amino acid sequence of SEQ ID NO:17. In some embodiments, the peptide has the amino acid sequence of SEQ ID NO:18. In some embodiments, the peptide has the amino acid sequence of SEQ ID NO:19. In some embodiments, the peptide has the amino acid sequence of SEQ ID NO:20. In some embodiments, the peptide has the amino acid sequence of SEQ ID NO:21. In some embodiments, the peptide has the amino acid sequence of SEQ ID NO:22. In some embodiments, the peptide has the amino acid sequence of SEQ ID NO:23. In some embodiments, the peptide has the amino acid sequence of SEQ ID NO:24. In some embodiments, the peptide has the amino acid sequence of SEQ ID NO:25. In some embodiments, the peptide has the amino acid sequence of SEQ ID NO:26. In some embodiments, the peptide has the amino acid sequence of SEQ ID NO:27. In some embodiments, the peptide has the amino acid sequence of SEQ ID NO:28. In some embodiments, the peptide has the amino acid sequence of SEQ ID NO:29. In some embodiments, the peptide has the amino acid sequence of SEQ ID NO:30. In some embodiments, the peptide has the amino acid sequence of SEQ ID NO:31. In some embodiments, the peptide has the amino acid sequence of SEQ ID NO:32. In some embodiments, the peptide has the amino acid sequence of SEQ ID NO:33. In some embodiments, the peptide has the amino acid sequence of SEQ ID NO:34. In some embodiments, the peptide has the amino acid sequence of SEQ ID NO:35. In some embodiments, the peptide has the amino acid sequence of SEQ ID NO:36. In some embodiments, the peptide has the amino acid sequence of SEQ ID NO:37. In some embodiments, the peptide has the amino acid sequence of SEQ ID NO:38. In some embodiments, the peptide has the amino acid sequence of SEQ ID NO:39. In some embodiments, the peptide has the amino acid sequence of SEQ ID NO:40. In some embodiments, the peptide has the amino acid sequence of SEQ ID NO:41. In some embodiments, the peptide has the amino acid sequence of SEQ ID NO:42. In some embodiments, the peptide has the amino acid sequence of SEQ ID NO:43. In some embodiments, the peptide has the amino acid sequence of SEQ ID NO:44. In some embodiments, the peptide has the amino acid sequence of SEQ ID NO:45. In some embodiments, the peptide has the amino acid sequence of SEQ ID NO:46. In some embodiments, the peptide has the amino acid sequence of SEQ ID NO:47. In some embodiments, the peptide has the amino acid sequence of SEQ ID NO:48. In some embodiments, the peptide has the amino acid sequence of SEQ ID NO:49. In some embodiments, the peptide has the amino acid sequence of SEQ ID NO:50. In some embodiments, the peptide has the amino acid sequence of SEQ ID NO:51. In some embodiments, the peptide has the amino acid sequence of SEQ ID NO:52. In some embodiments, the peptide has the amino acid sequence of SEQ ID NO:53. In some embodiments, the peptide has the amino acid sequence of SEQ ID NO:54. In some embodiments, the peptide has the amino acid sequence of SEQ ID NO:55. In some embodiments, the peptide has the amino acid sequence of SEQ ID NO:56. In some embodiments, the peptide has the amino acid sequence of SEQ ID NO:57. In some embodiments, the peptide has the amino acid sequence of SEQ ID NO:58. In some embodiments, the peptide has the amino acid sequence of SEQ ID NO:59. In some embodiments, the peptide has the amino acid sequence of SEQ ID NO:60. In some embodiments, the peptide has the amino acid sequence of SEQ ID NO:61. In some embodiments, the peptide has the amino acid sequence of SEQ ID NO:62. In some embodiments, the peptide has the amino acid sequence of SEQ ID NO:63. In some embodiments, the peptide has the amino acid sequence of SEQ ID NO:64. In some embodiments, the peptide has the amino acid sequence of SEQ ID NO:65. In some embodiments, the peptide has the amino acid sequence of SEQ ID NO:66. In some embodiments, the peptide has the amino acid sequence of SEQ ID NO:67. In some embodiments, the peptide has the amino acid sequence of SEQ ID NO:68, In some embodiments, the peptide has the amino acid sequence of SEQ ID NO:69. In some embodiments, the peptide has the amino acid sequence of SEQ ID NO:70. In some embodiments, the peptide has the amino acid sequence of SEQ ID NO:71. In some embodiments, the peptide has the amino acid sequence of SEQ ID NO:72. In some embodiments, the peptide has the amino acid sequence of SEQ ID NO:73. In some embodiments, the peptide has the amino acid sequence of SEQ ID NO:74. In some embodiments, the peptide has the amino acid sequence of SEQ ID NO:75. In some embodiments, the peptide has the amino acid sequence of SEQ ID NO:76. In some embodiments, the peptide has the amino acid sequence of SEQ ID NO:77. In some embodiments, the peptide has the amino acid sequence of SEQ ID NO:78. In some embodiments, the peptide has the amino acid sequence of SEQ ID NO:79. In some embodiments, the peptide has the amino acid sequence of SEQ ID NO:80. In some embodiments, the peptide has the amino acid sequence of SEQ ID NO:81. In some embodiments, the peptide has the amino acid sequence of SEQ ID NO:82. In some embodiments, the peptide has the amino acid sequence of SEQ ID NO:83. In some embodiments, the peptide has the amino acid sequence of SEQ ID NO:84. In some embodiments, the peptide has the amino acid sequence of SEQ ID NO:85. In some embodiments, the peptide has the amino acid sequence of SEQ ID NO:86. In some embodiments, the peptide has the amino acid sequence of SEQ ID NO:87. In some embodiments, the peptide has the amino acid sequence of SEQ ID NO:88. In some embodiments, the peptide has the amino acid sequence of SEQ ID NO:89. In some embodiments, the peptide has the amino acid sequence of SEQ ID NO:90. In some embodiments, the peptide has the amino acid sequence of SEQ ID NO:91. In some embodiments, the peptide has the amino acid sequence of SEQ ID NO:92. In some embodiments, the peptide has the amino acid sequence of SEQ ID NO:93. In some embodiments, the peptide has the amino acid sequence of SEQ ID NO:94. In some embodiments, the peptide has the amino acid sequence of SEQ ID NO:95. In some embodiments, the peptide has the amino acid sequence of SEQ ID NO:96. In some embodiments, the peptide has the amino acid sequence of SEQ ID NO:97. In some embodiments, the peptide has the amino acid sequence of SEQ ID NO:98. In some embodiments, the peptide has the amino acid sequence of SEQ ID NO:99. In some embodiments, the peptide has the amino acid sequence of SEQ ID NO:100. In some embodiments, the peptide has the amino acid sequence of SEQ ID NO:101. In some embodiments, the peptide has the amino acid sequence of SEQ ID NO:102. In some embodiments, the peptide has the amino acid sequence of SEQ ID NO:103. In some embodiments, the peptide has the amino acid sequence of SEQ ID NO:104. In some embodiments, the peptide has the amino acid sequence of SEQ ID NO:105. In some embodiments, the peptide has the amino acid sequence of SEQ ID NO:106. In some embodiments, the peptide has the amino acid sequence of SEQ ID NO:107. In some embodiments, the peptide has the amino acid sequence of SEQ ID NO:108. In some embodiments, the peptide has the amino acid sequence of SEQ ID NO:109. In some embodiments, the peptide has the amino acid sequence of SEQ ID NO:110. In some embodiments, the peptide has the amino acid sequence of SEQ ID NO:111. In some embodiments, the peptide has the amino acid sequence of SEQ ID NO:112. In some embodiments, the peptide has the amino acid sequence of SEQ ID NO:113. In some embodiments, the peptide has the amino acid sequence of SEQ ID NO:114. In some embodiments, the peptide has the amino acid sequence of SEQ ID NO:115. In some embodiments, the peptide has the amino acid sequence of SEQ ID NO:116. In some embodiments, the peptide has the amino acid sequence of SEQ ID NO:117. In some embodiments, the peptide has the amino acid sequence of SEQ ID NO:118. In some embodiments, the peptide has the amino acid sequence of SEQ ID NO:119.

### EXAMPLES

The following examples are not intended to limit the scope of what the inventors regard as their invention nor are they intended to represent that the experiments below are all or the only experiments performed.

### Example 1

### Elispot Assessment of Immune Responses to hTERT Peptides by Peripheral Blood-Derived T Cells after hTERT-DC Administration

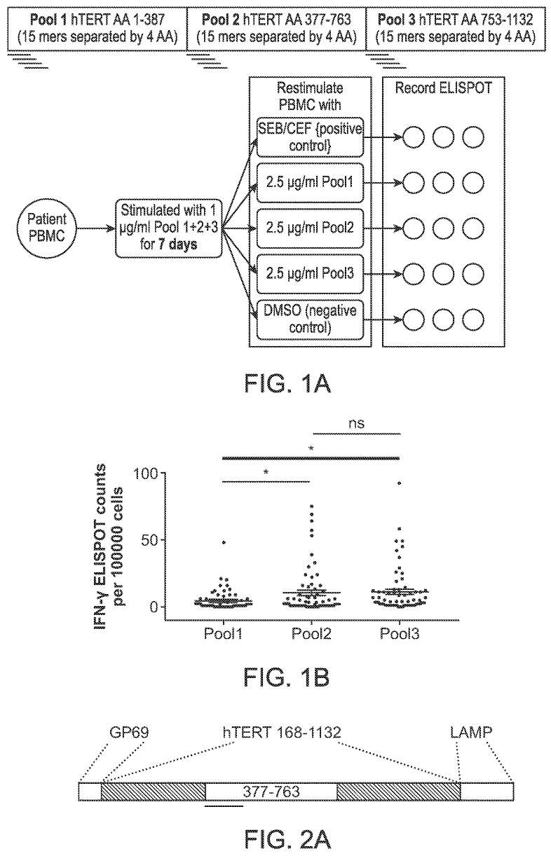

PBMCs were collected from patients enrolled in the AST-VAC1 Phase 2 clinical trial (Khoury M D et al., 2017), and the hTERT specific T cell responses in PBMCs were assessed by enzyme-linked immunospot assay (ELISPOT) analysis. For this analysis, PBMCs were collected: 1) pre-hTERT-DC vaccination; 2) after the third and sixth primary hTERT-DC vaccination; 3) after the 4 week rest period; 4) and after hTERT-DC boosts 1, 3, 5, and 6. Patient PBMC samples were stimulated with 1 μg/mL each of three hTERT peptide pools and cultured in AIM-V media (Life Technologies) containing 5% human AB serum (Valley Biomedical) for 7 days at 37° C., 5% CO2. The three hTERT peptide pools consisted of 281 overlapping peptides (−94 per pool) that spanned the entire hTERT protein. hTERT peptide pool 1 spanned hTERT amino acids 1-387; pool 2 spanned hTERT amino acids 377-763 and pool 3 spanned hTERT amino acids 753-1132 (FIG. 1A).

PBMCs were transferred into multiscreen-IP PVDF ELISPOT plates (Millipore) coated with 10 μg/mL of mouse anti-human IFN-γ mAb (MabTech) and restimulated with mock (DMSO alone) or one of the individual hTERT peptide pools (2.5 μg/mL) for 16-24 hours in media at 37° C., 5% CO2. The development of IFN-γ spots was followed based on the manufacturer's instructions (MabTech). Briefly, cells were removed and plates were washed in 1× PBS (Life Technologies). Biotinylated detection antibody (MabTech) was added to each well at 1 μg/mL for 2 hours at room temperature. After washing the plates, streptavidin-ALP (MabTech) at a 1:1000 dilution was added and incubated for 1 hour at room temperature. Spots were developed by adding NBT/BCIP (Moss). An ImmunoSpot Analyzer (Cellular Technology, Ltd) was used to quantitate the number of spots detected. The frequency of T cells specific for each hTERT peptide pool was determined by subtracting the mock control spots from the number of spots observed in response to the hTERT peptide pools. A patient was considered to be positive for an hTERT T cell immune response if a 2.5 fold or greater change in the number of IFN-γ spots as assessed by ELISPOT assay for at least one of the three hTERT peptide pools was observed post-vaccination compared to pre-vaccination levels. A minimum of 35 spots per 1×10⁶ cells was required. Results indicated that the maximal T cell response in these assays was detected in pools 2 and 3 (FIG. 1B).

### Example 2

### Computationally Deduced Peptide Pools That Potentiate hTERT-Specific T Cell Response

Further studies were conducted to identify the individual hTERT peptide epitopes recognized by the hTERT specific T cells found after the hTERT-DC administration. Due to limited availability of patient PBMCs from any particular patient at a given time point, the follow-up assays were focused on particular 15-mers that were likely to generate an immune response. To achieve this, selection methods and accompanying computation algorithms as described below were used.

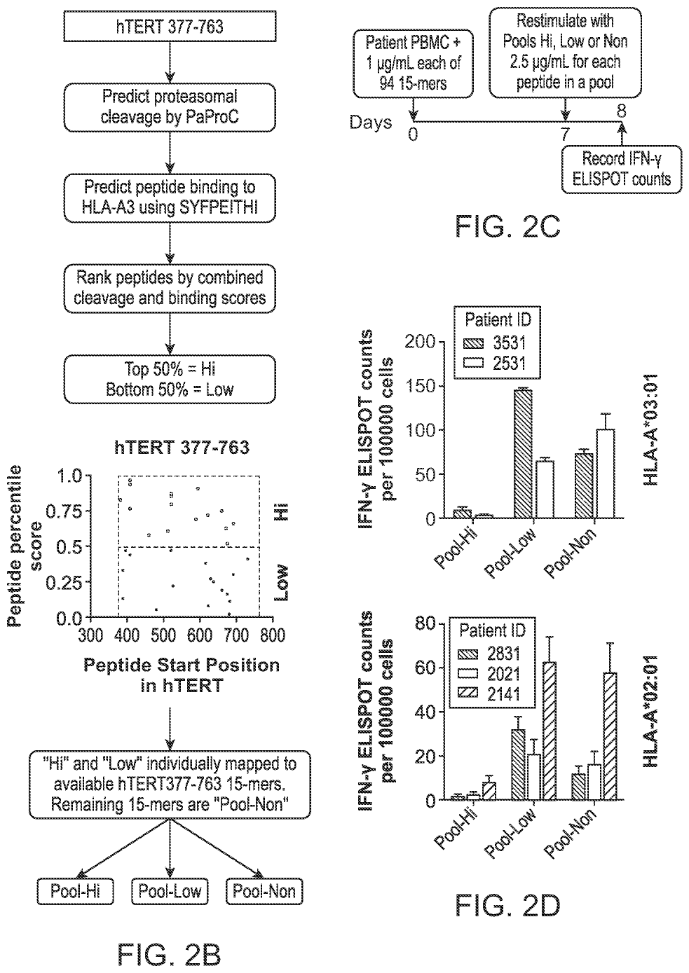

Subjects expressing the HLA-A*03:01 allele or the HLA-A*02:01 allele (the most commonly represented MHC alleles present among patients who exhibited hTERT-specific immune response) were selected for the follow-up analysis. Peptides derived from hTERT amino acids 377-763 or pool #2 were used, as these exhibited maximal immune response above pre-immunized levels. Computational methods were used to predict HLA-A*03:01 hTERT peptides or HLA-A*02:01 hTERT peptides from the hTERT377-763 amino acid sequence as follows. First, proteasomal analysis using PaProC (Kuttler, C., et al., An algorithm for the prediction of proteasomal cleavages. J Mol Biol, 2000. 298(3): p. 417-29; Nussbaum, A. K., et al., PAProC: a prediction algorithm for proteasomal cleavages available on the WWW. Immunogenetics, 2001. 53(2): p. 87-94a) was performed to deduce possible peptides cleaved from hTERT377-763. The SYFPEITHI database (Rammensee, H., et al., SYFPEITHI: database for MHC ligands and peptide motifs. Immunogenetics, 1999. 50(3-4): p. 213-9) was then used to predict binding affinity of HLA-A*03:01 or HLA-A*02:01 for these peptides. A combined binding and cleavage score was assigned using the MAPPP bioinformatics tool (Hakenberg, J., et al., MAPPP: MHC class I antigenic peptide processing prediction. Appl Bioinformatics, 2003. 2(3): p. 155-8). For each allele, i.e. HLA-A*03:01 and HLA-A*02:01, scored peptides were ranked into top 50% (Hi) and bottom 50% (Low), and then individually mapped to 15-mers from pool 2 covering hTERT377-763. 15-mers mapped to “Hi” and “Low” comprised the hTERT peptide pools defined as “Pool-Hi” and “Pool-Low”, respectively. All hTERT 15-mer peptides that were not identified by the computational analysis were collectively defined as “Pool-non”. The “Pool-non” group could still contain hTERT peptides that bind to HLA-A*03:01 or HLA-A*02:01, but were missed by one or more of the computational algorithms. FIG. 2B depicts the computational process.

Pooled “Hi”, “Low” and “Non” peptides were used in ELISPOT assays to assess which peptides stimulated hTERT-specific T cells from HLA-A*03:01 positive patients or HLA-A*02:01 positive patients. For these ELISPOT assays, cryopreserved PBMCs were thawed and stimulated for 7 days in AIM-V medium (Gibco) substituted with 5% human AB serum (Valley biomedical) with 1 μg/mL each of 94 15-mer peptides covering hTERT377-763. The cells were subsequently restimulated for 24 hours with peptide pools “Hi”, “Low”, or “Non” at 2.5 μg/mL of each individual peptide within a pool (FIG. 2C). For negative controls, cells alone without additional peptides at day 7 were used, and for positive controls staphylococcal enterotoxin B (SEB, Sigma-Aldrich) or peptides derived from cytomegalovirus, Epstein-Barr virus and Influenza virus (CEF, C.T.L.) were used. All conditions were performed in triplicate wells. IFN-γ ELISPOT counts per 100,000 cells were recorded and normalized by subtracting the counts of the negative control wells. The results are depicted in FIG. 2D. Surprisingly, the majority of targeted peptides exhibiting positive hTERT-specific T cell responses were predicted to have low affinity for HLA-A*03:01. Similar results were found in the analysis of hTERT specific T cells from subjects in the trial carrying the HLA-A*02:01 allele. This finding may be related to the fact that hTERT is a self-antigen and T cells specific to high affinity epitopes may be deleted during development by negative selection.

### Example 3

### Identification and Analysis of Peptide Epitopes Targeted by hTERT-Specific T Cells

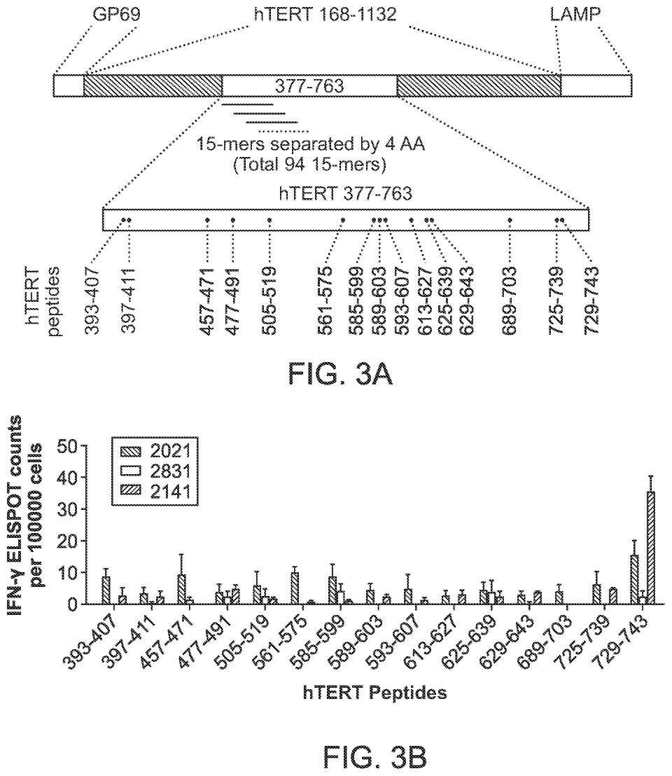

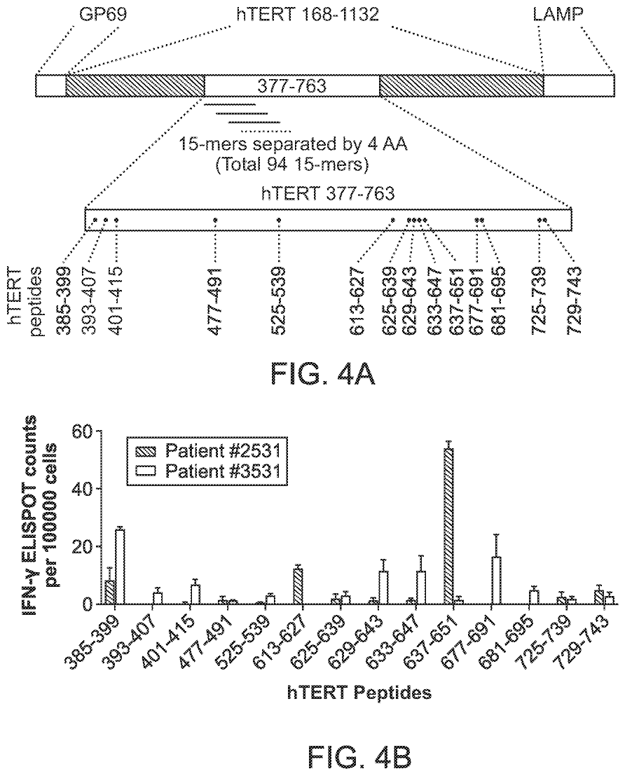

In order to delineate single peptides and peptide epitopes that can potentiate an hTERT-specific T cell response, individual peptides from the “pool-low” for both MEW haplotypes HLA-A*02:01 and HLA-A*03:01 were used. Tables 1 and 2 show the peptides for “pool-low” for HLA-A*02:01 and HLA-A*03:01, respectively. These peptides are also mapped to the hTERT377-763 region in FIGS. 3A and 4A for HLA-A*02:01 and HLA-A*03:01, respectively.

The ELISPOT assay was repeated. Cryopreserved patient PBMCs were thawed and stimulated for 7 days with 1 μg/mL each of 94 pool #2 15-mer peptides. The cells were subsequently restimulated for 24 hours with individual peptides from pool low at 2.5 IFN-γ ELISPOT counts per 100,000 cells were recorded. Data was normalized to control wells with cells that were not restimulated with peptides at day 7 (FIG. 3 and FIG. 4). From these assays it was observed that hTERT637-651 (DYVVGARTFRREKRA (SEQ ID NO:1)) and hTERT385-399 (RYWQMRPLFLELLGN (SEQ ID NO:2)) were the peptides that modulated the most potent hTERT-specific response in the context of HLA-A*03:01 and hTERT729-743 (IASIIKPQNTYCVRR (SEQ ID NO:3)) modulated the most potent hTERT-specific immune response in the context of HLA-A*02:01.

TABLE 1
Amino acid sequences of individual hTERT
peptides from Pool-Low for HLA-A*02:01.
The hTERT peptide sequence numbering in
the table refers to the full length protein
sequence of human telomerase reverse
transcriptase isoform 1, NCBI Accession
NO: NP_937983.2

| hTERT | |
|---|---|
| Peptide | Sequence |
| 393-407 | FLELLGNHAQCPYGV (SEQ ID NO: 4) |
| 397-411 | LGNHAQCPYGVLLKT (SEQ ID NO: 5) |
| 457-471 | SPWQVYGFVRACLRR (SEQ ID NO: 6) |
| 477-491 | LWGSRHNERRFLRNT (SEQ ID NO: 7) |
| 505-519 | LQELTWKMSVRDCAW (SEQ ID NO: 8) |
| 561-575 | FYVTETTFQKNRLFF (SEQ ID NO: 9) |
| 585-599 | QSIGIRQHLKRVQLR (SEQ ID NO: 10) |
| 589-603 | IRQHLKRVQLRELSE (SEQ ID NO: 11) |
| 593-607 | LKRVQLRELSEAEVR (SEQ ID NO: 12) |
| 613-627 | RPALLTSRLRFIPKP (SEQ ID NO: 13) |
| 625-639 | PKPDGLRPIVNMDYV (SEQ ID NO: 14) |
| 629-643 | GLRPIVNMDYVVGAR (SEQ ID NO: 15) |
| 689-703 | AWRTFVLRVRAQDPP (SEQ ID NO: 16) |
| 725-739 | LTEVIASIIKPQNTY (SEQ ID NO: 17) |
| 729-743 | IASIIKPQNTYCVRR (SEQ ID NO: 18) |

TABLE 2
Amino acid sequences of individual hTERT
peptides from Pool-Low for HLA-A*03:01.
The hTERT peptide sequence numbering in
the table refers to the full length protein
sequence of human telomerase reverse
transcriptase isoform 1, NCBI Accession
NO: NP_937983.2

| hTERT | |
|---|---|
| Peptide | Sequence |
| 385-399 | RYWQMRPLFLELLGN (SEQ ID NO: 19) |
| 393-407 | FLELLGNHAQCPYGV (SEQ ID NO: 20) |
| 401-415 | AQCPYGVLLKTHCPL (SEQ ID NO: 21) |
| 477-491 | LWGSRHNERRFLRNT (SEQ ID NO: 22) |
| 525-539 | GVGCVPAAEHRLREE (SEQ ID NO: 23) |
| 613-627 | RPALLTSRLRFIPKP (SEQ ID NO: 24) |
| 625-639 | PKPDGLRPIVNMDYV (SEQ ID NO: 25) |
| 629-643 | GLRPIVNMDYVVGAR (SEQ ID NO: 26) |
| 633-647 | IVNMDYVVGARTFRR (SEQ ID NO: 27) |
| 637-651 | DYVVGARTFRREKRA (SEQ ID NO: 28) |
| 677-691 | GASVLGLDDIHRAWR (SEQ ID NO: 29) |
| 681-695 | LGLDDIHRAWRTFVL (SEQ ID NO: 30) |
| 725-739 | LTEVIASIIKPQNTY (SEQ ID NO: 31) |
| 729-743 | IASIIKPQNTYCVRR (SEQ ID NO: 32) |

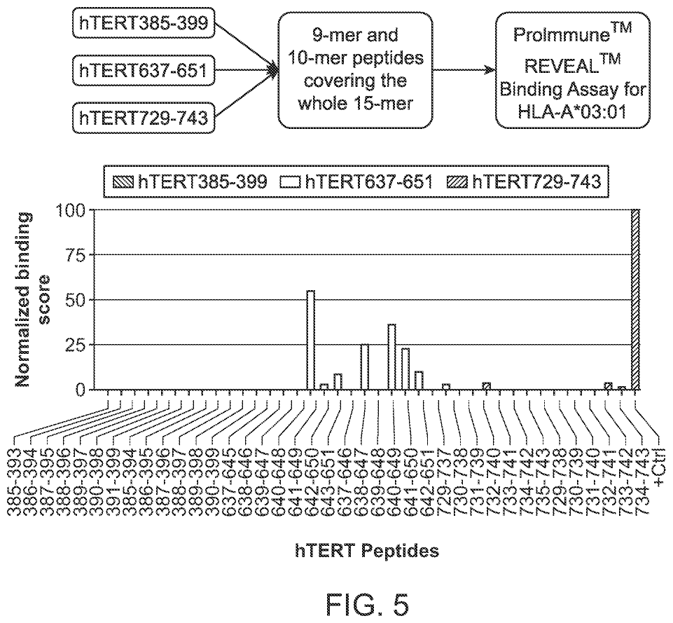

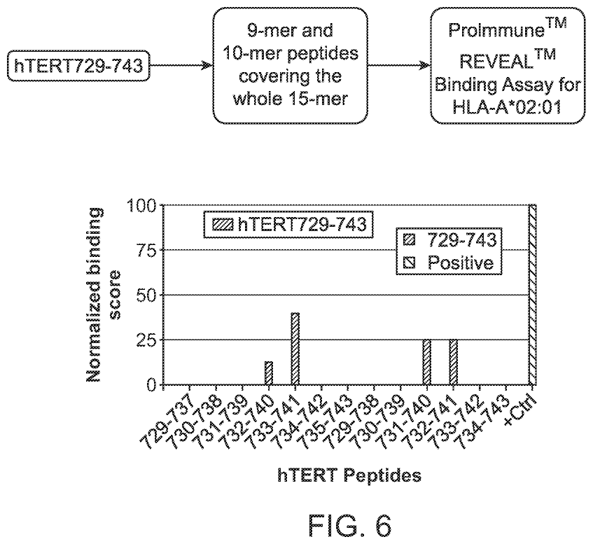

To further delineate the identity of individual peptides and peptide epitopes that potentiate the hTERT-specific T cell response, 9-10-mer peptides were derived from the 15 mer peptides that exhibited potent immune responses, and the binding affinity of the 9-10 mers to HLA-A*02:01 and HLA-A*03:01 was measured. The 9 and 10 amino acid sequences were chosen to cover the entire 15 amino acid sequence for each 15-mer peptide, and are shown in Table 3. The REVEAL™ assay (ProImmune Inc.) was used to ascertain the binding affinity of the 9-10 mer peptides to HLA-A*03:01 and HLA-A*02:01. Briefly, the peptides were first synthesized to over 70% purity and tested by mass spectrometry. Subsequently, the binding affinity of these peptides was determined as a binding score normalized to the amount of binding observed for known positive control peptides to HLA*03:01 (FIG. 5) or HLA*02:01 (FIG. 6). HLA-A*03:01 peptides hTERT639-647, hTERT638-647, hTERT643-651, and hTERT639-648 had binding scores above 20 (FIG. 5), indicative of stable binding to HLA-A*03:01 to form pMHC complex. These peptides were derived from hTERT637-651. Additionally, hTERT641-649 and hTERT640-649 exhibited binding scores −10, indicative of at least transient binding to HLA-A*03:01. 9-mer and 10-mer peptides derived from hTERT385-399 and hTERT729-743 did not show appreciable binding affinity for HLA-A*03:01. For HLA-A*02:01 It was observed that peptides hTERT733-741, hTERT731-740, and hTERT732-741 had binding scores above 20 (FIG. 6), indicative of stable binding to HLA-A*02:01 to form pMHC complex. These peptides were derived from hTERT729-743. Additionally, hTERT732-740 exhibited binding score >10, indicative of at least transient binding to HLA-A*02:01.

TABLE 3
Amino acid sequences of individual 9-mer
and 10-mer peptides derived from hTERT385-399,
hTERT637-651 and hTERT729-743. The hTERT
peptide sequence numbering in the table refers
to the full length protein sequence of human
telomerase reverse transcriptase isoform 1,
NCBI Accession NO: NP_937983.2

| hTERT | |
|---|---|
| Peptide | Sequence |
| 385-393 | RYWQMRPLF (SEQ ID NO: 33) |
| 386-394 | YWQMRPLFL (SEQ ID NO: 34) |
| 387-395 | WQMRPLFLE (SEQ ID NO: 35) |
| 388-396 | QMRPLFLEL (SEQ ID NO: 36) |
| 389-397 | MRPLFLELL (SEQ ID NO: 37) |
| 390-398 | RPLFLELLG (SEQ ID NO: 38) |
| 391-399 | PLFLELLGN (SEQ ID NO: 39) |
| 385-394 | RYWQMRPLFL (SEQ ID NO: 40) |
| 386-395 | YWQMRPLFLE (SEQ ID NO: 41) |
| 387-396 | WQMRPLFLEL (SEQ ID NO: 42) |
| 388-397 | QMRPLFLELL (SEQ ID NO: 43) |
| 389-398 | MRPLFLELLG (SEQ ID NO: 44) |
| 390-399 | RPLFLELLGN (SEQ ID NO: 45) |
| 385-393 | RYWQMRPLF (SEQ ID NO: 46) |
| 637-645 | DYVVGARTF (SEQ ID NO: 47) |
| 638-646 | YVVGARTFR (SEQ ID NO: 48) |
| 639-647 | VVGARTFRR (SEQ ID NO: 49) |
| 640-648 | VGARTFRRE (SEQ ID NO: 50) |
| 641-649 | GARTFRREK (SEQ ID NO: 51) |
| 642-650 | ARTFRREKR (SEQ ID NO: 52) |
| 643-651 | RTFRREKRA (SEQ ID NO: 53) |
| 637-646 | DYVVGARTFR (SEQ ID NO: 54) |
| 638-647 | YVVGARTFRR (SEQ ID NO: 55) |
| 639-648 | VVGARTFRRE (SEQ ID NO: 56) |
| 640-649 | VGARTFRREK (SEQ ID NO: 57) |
| 641-650 | GARTFRREKR (SEQ ID NO: 58) |
| 642-651 | ARTFRREKRA (SEQ ID NO: 59) |
| 729-737 | IASIIKPQN (SEQ ID NO: 60) |
| 730-738 | ASIIKPQNT (SEQ ID NO: 61) |
| 731-739 | SIIKPQNTY (SEQ ID NO: 62) |
| 732-740 | IIKPQNTYC (SEQ ID NO: 63) |
| 733-741 | IKPQNTYCV (SEQ ID NO: 64) |
| 734-742 | KPQNTYCVR (SEQ ID NO: 65) |
| 735-743 | PQNTYCVRR (SEQ ID NO: 66) |
| 729-738 | IASIIKPQNT (SEQ ID NO: 67) |
| 730-739 | ASIIKPQNTY (SEQ ID NO: 68) |
| 731-740 | SIIKPQNTYC (SEQ ID NO: 69) |
| 732-741 | IIKPQNTYCV (SEQ ID NO: 70) |
| 733-742 | IKPQNTYCVR (SEQ ID NO: 71) |

TABLE 4
Modified hTERT-specific peptides for HLA-A*02:01.
The original peptide sequence and position numbering
in the table refers to the full length protein
sequence of human telomerase reverse transcriptase
isoform 1, NCBI Accession NO: NP_937983.2

| Original | | 100% | Binding | Original |
|---|---|---|---|---|
| peptide and | | BLAST | Score | binding |
| position | Modified | Match? | (SYFPEITHI) | Score |
| IKPQNTYCV | ILPQNTYCI (SEQ ID NO: 72) | No | 23 | 15 |
| 733-741 | ILPQNTYCV (SEQ ID NO: 73) | No | 25 | 15 |
| (SEQ ID | ILPQNTYCL (SEQ ID NO: 74) | No | 25 | 15 |
| NO: 64) | | | | |
| | IMPQNTYCI (SEQ ID NO: 75) | No | 21 | 15 |
| | IMPQNTYCV (SEQ ID | No | 23 | 15 |
| | NO: 76) | | | |
| | IMPQNTYCL (SEQ ID | No | 23 | 15 |
| | NO: 77) | | | |
| SIIKPQNTYC | SLIKPQNTYI (SEQ ID | No | 22 | 12 |
| | NO: 78) | | | |
| 731-740 | SLIKPQNTYV (SEQ ID | No | 24 | 12 |
| | NO: 79) | | | |
| (SEQ ID | SLIKPQNTYL (SEQ ID | No | 24 | 12 |
| NO: 69) | NO: 80) | | | |
| | SMIKPQNTYI (SEQ ID | No | 20 | 12 |
| | NO: 81) | | | |
| | SMIKPQNTYV (SEQ ID | No | 22 | 12 |
| | NO: 82) | | | |
| | SMIKPQNTYL (SEQ ID | No | 22 | 12 |
| | NO: 83) | | | |
| IIKPQNTYCV | ILKPQNTYCI (SEQ ID | No | 22 | 22 |
| | NO: 84) | | | |
| 732-741 | ILKPQNTYCV (SEQ ID | No | 24 | 22 |
| | NO: 85) | | | |
| (SEQ ID | ILKPQNTYCL (SEQ ID | No | 24 | 22 |
| NO: 70) | NO: 86) | | | |
| | IMKPQNTYCI (SEQ ID | No | 20 | 22 |
| | NO: 87) | | | |
| | IMKPQNTYCV (SEQ ID | No | 22 | 22 |
| | NO: 88) | | | |
| | IMKPQNTYCL (SEQ ID | No | 22 | 22 |
| | NO: 89) | | | |
| IIKPQNTYC | ILKPQNTYI (SEQ ID NO: 90) | No | 21 | 11 |
| 732-740 | ILKPQNTYV (SEQ ID | No | 23 | 11 |
| | NO: 91) | | | |
| (SEQ ID | ILKPQNTYL (SEQ ID NO: 92) | No | 23 | 11 |
| NO: 63) | | | | |
| | IMKPQNTYI (SEQ ID NO: 93) | No | 19 | 11 |
| | IMKPQNTYV (SEQ ID | No | 21 | 11 |
| | NO: 94) | | | |
| | IMKPQNTYL (SEQ ID | No | 21 | 11 |
| | NO: 95) | | | |

TABLE 5
Modified hTERT-specific peptides for HLA-A*03:01. The original
peptide sequence and position numbering in the table refers to
the full length protein sequence of human telomerase reverse
transcriptase isoform 1, NCBI Accession NO: NP_937983.2

| | | Naturally | | Predicted |
|---|---|---|---|---|
| | | occurring? | Predicted | binding |
| Original | | 100% | Binding | score of |
| peptide | | BLAST | Score | Original |
| and position | Modified | Match | (SYFPEITHI) | peptide |
| VVGARTFRR | VLGARTFRK (SEQ ID | Yes | 24 | 18 |
| | NO: 96) | | | |
| 639-647 | VLGARTFRR (SEQ ID | Yes | 18 | 18 |
| | NO: 97) | | | |
| (SEQ ID NO: 49) | VIGARTFRK (SEQ ID | No | 22 | 18 |
| | NO: 98) | | | |
| | VIGARTFRR (SEQ ID | No | 16 | 18 |
| | NO: 99) | | | |
| YVVGARTFRR | YLVGARTFRK (SEQ ID | No | 22 | 16 |
| | NO: 100) | | | |
| 638-647 | YLVGARTFRR (SEQ ID | No | 16 | 16 |
| | NO: 101) | | | |
| (SEQ ID NO: 55) | YIVGARTFRK (SEQ ID | No | 20 | 16 |
| | NO: 102) | | | |
| | YIVGARTFRR (SEQ ID | Yes | 14 | 16 |
| | NO: 103) | | | |
| RTFRREKRA | RLFRREKRK (SEQ ID | No | 29 | 9 |
| | NO: 104) | | | |
| 643-651 | RLFRREKRR (SEQ ID | Yes | 23 | 9 |
| | NO: 105) | | | |
| (SEQ ID NO: 53) | RIFRREKRK (SEQ ID | No | 27 | 9 |
| | NO: 106) | | | |
| | RIFRREKRR (SEQ ID | No | 21 | 9 |
| | NO: 107) | | | |
| VVGARTFRRE | VLGARTFRRK (SEQ ID | No | 22 | 12 |
| | NO: 108) | | | |
| 639-648 | VLGARTFRRR (SEQ ID | No | 16 | 12 |
| | NO: 109) | | | |
| (SEQ ID NO: 56) | VIGARTFRRK (SEQ ID | No | 20 | 12 |
| | NO: 110) | | | |
| | VIGARTFRRR (SEQ ID | No | 14 | 12 |
| | NO: 111) | | | |
| VGARTFRREK | VLARTFRREK (SEQ ID | No | 25 | 15 |
| | NO: 112) | | | |
| 640-649 | VLARTFRRER (SEQ ID | No | 19 | 15 |
| | NO: 113) | | | |
| (SEQ ID NO: 57) | VIARTFRREK (SEQ ID | No | 23 | 15 |
| | NO: 114) | | | |
| | VIARTFRRER (SEQ ID | No | 17 | 15 |
| | NO: 115) | | | |
| GARTFRREK | GLRTFRREK (SEQ ID | No | 24 | 14 |
| | NO: 116) | | | |
| 641-649 | GLRTFRRER (SEQ ID | No | 18 | 14 |
| | NO: 117) | | | |
| (SEQ ID NO: 51) | GIRTFRREK (SEQ ID | No | 22 | 14 |
| | NO: 118) | | | |
| | GIRTFRRER (SEQ ID | No | 16 | 14 |
| | NO: 119) | | | |

What is claimed is:

1. A method for eliciting a cell-mediated immune response in a human subject, comprising administering to the subject an effective amount of an immunotherapeutic composition that stimulates an immune response to one or more peptides selected from peptides having the amino acid sequence of any one of SEQ ID NO:4-119.

2. The method according to claim 1, wherein the immunotherapeutic composition comprises antigen-presenting cells that present on their surface a peptide selected from peptides having the amino acid sequence of any one of SEQ ID NO:4-119.

3. The method according to claim 2, wherein the antigen-presenting cells are transfected or pulsed with mRNA encoding one or more peptides selected from peptides having the amino acid sequence of any one of SEQ ID NO:4-119.

4. The method according to claim 2 or 3, wherein the antigen-presenting cells are dendritic cells, macrophages, B cells, or a combination thereof.

5. The method according to claim 4, wherein the antigen-presenting cells are mature dendritic cells.

6. The method according to any one of claims 1-5, wherein the subject is in need of immunotherapy.

7. The method according to claim 6, wherein the subject has cancer.

8. The method according to claim 7, wherein the subject has acute myeloid leukemia (AML).

9. The method according to claim 3, wherein the mRNA comprises a construct encoding two or more copies of any of the one or more peptides selected from peptides having the amino acid sequence of any one of SEQ ID NO:4-119.

10. The method according to any one of claims 2-9, wherein the antigen-presenting cells are derived from the subject.

11. The method according to any one of claims 2-9, wherein the antigen-presenting cells are derived from pluripotent stem cells.

12. The method according to claim 11, wherein the pluripotent stem cells are human embryonic stem cells.

13. An immunotherapeutic composition that stimulates an immune response to one or more peptides selected from peptides having the amino acid sequence of any one of SEQ ID NO:4-119.

14. The composition of claim 13, wherein the immunotherapeutic composition comprises antigen-presenting cells that present on their surface a peptide selected from the peptides having the amino acid sequence of any one of SEQ ID NO:4-119.

15. The composition of claim 14, wherein the antigen-presenting cells are transfected or pulsed with mRNA encoding one or more peptides selected from the peptides having the amino acid sequence of any one of SEQ ID NO:4-119.

16. The composition according to any of claims 13 to 15, wherein the antigen-presenting cells are dendritic cells, macrophages, B cells, or a combination thereof.

17. The composition of claim 16, wherein the antigen-presenting cells are mature dendritic cells.

18. The composition according to any of claims 15 to 17, wherein the mRNA comprises a construct encoding two or more copies of any of the one or more peptides selected from the peptides having the amino acid sequence of any one of SEQ ID NO:4-119.

19. An antigen-presenting cell presenting on its surface a peptide selected from the peptides having the amino acid sequence of any one of SEQ ID NO:4-119.

20. An antigen-presenting cell transfected or pulsed with mRNA encoding one or more peptides selected from the peptides having the amino acid sequence of any one of SEQ ID NO:4-119.

21. The cell according to claim 19 or 20, wherein the antigen-presenting cells are dendritic cells, macrophages, B cells, or a combination thereof.

22. The cell of claim 19 or 20, wherein the cell is a dendritic cell.

23. The cell of claim 22, wherein the mRNA comprises a construct encoding two or more copies of any of the one or more peptides having the amino acid sequence of any one of SEQ ID NO:4-119.

24. The cell of claim 22, wherein the transfection is done by electroporation.

25. The cell of claim 22, wherein the mRNA construct further comprises a lysosomal targeting sequence.

26. An in vitro complex comprising a MHC Class I molecule complexed with a peptide chosen from the peptides having the amino acid sequence of any one of SEQ ID NO:4-119.

27. The in vitro complex of claim 26, wherein the MHC Class I molecule is encoded by the HLA-A locus.

28. The in vitro complex of claim 26 or 27, further comprising a conjugated biotin molecule.
